(19) United States
(12) Patent Application Publication
(10) Pub. No.: US 2024/0238385 A1
(43) Pub. Date: Jul. 18, 2024

(54) ISOLATED OR ARTIFICIAL NUCLEOTIDE SEQUENCES FOR USE IN NEURODEGENERATIVE DISEASES

(71) Applicant: Universidade do Algarve, Faro (PT)

(72) Inventors: Clévio David RODRIGUES NÓBREGA, Faro (PT); Rebekah COVACO KOPPENOL, Faro (PT); Adriana Isabel DO VALE MARCELO, Caxarias (PT); André Filipe VIEIRA DA CONCEIÇÃO, Silves (PT)

(21) Appl. No.: 18/559,936

(22) PCT Filed: May 13, 2022
(86) PCT No.: PCT/IB2022/054493

§ 371 Date: Nov. 9, 2023

(30) Foreign Application Priority Data
May 13, 2021 (PT) 117230

(51) Int. Cl. A61K 38/46; A61K 48/00

(52) U.S. Cl. CPC A61K 38/46; A61K 48/0033; A61K 48/005

(57) ABSTRACT

The present disclosure relates to an isolated or artificial nucleotide sequence encoding the GTPase-activating protein-binding protein 1 (G3BP1), for use in medicine, preferably in the treatment of polyglutamine diseases. Furthermore, the present invention is also related to a vector comprising such sequence, a host cell comprising such vector, a protein G3BP1, or a composition thereof, for use for use in medicine, preferably in the treatment of polyglutamine diseases.

### TECHNICAL FIELD

The present disclosure relates to isolated or artificial nucleotide sequences encoding the GTPase-activating protein-binding protein 1 (G3BP1), for use in medicine, preferably in the treatment of polyglutamine diseases. Furthermore, the present invention is also related to a vector comprising such sequence, a host cell comprising such vector, a protein G3BP1, or a composition thereof, for use for use in medicine, preferably in the treatment of polyglutamine diseases.

### BACKGROUND

Polyglutamine (PolyQ) diseases are a group of hereditary neurodegenerative diseases including Huntington's disease (HD), Spinal bulbar muscular atrophy (SBMA), Dentatorubral-pallidoluysian atrophy (DRPLA), and several spinocerebellar ataxias (SCA1, 2, 3, 6, 7, and 17). These diseases are characterized by abnormal expansions of the trinucleotide CAG in coding regions of each disease-associated gene, which encode for an expanded polyglutamine tract in the respective proteins. A central feature of these diseases is the aggregation of the mutant protein, which promotes aberrant interactions with other proteins and mRNAs, leading to the impairment of several cellular pathways and organelles¹. Nevertheless, the complete picture of the molecular events leading to selective neurodegeneration of specific brain region is yet to be fully understood. Moreover, until now there are no therapies able to stop or delay the disease progression that culminates in the premature death of patients with PolyQ diseases.

SCA2 and SCA3 (or Machado-Joseph disease—MJD) are two of the most prevalent spinocerebellar ataxias, being both characterized by a neurodegenerative profile that mainly affects the cerebellum and the brain stem. SCA2 is caused by an abnormal mutation in the ATXN2 gene above 31-33 CAG repeats, resulting in an overexpanded ataxin-2 protein2. SCA3 is caused by an abnormal mutation above 44-45 CAG in the ATXN3 gene, causing the ataxin-3 protein to be abnormally expanded3,4. Both mutant ataxin-2 and ataxin-3, are prone to aggregate and form large inclusions capable of sequestering other proteins. Though large inclusions are often reported as hallmarks of the disease, whether they are directly leading to toxicity is still a matter of debate⁵⁻⁸.

The pathological aggregation of polyQ proteins and the abnormal interactions in which they engage can result in significant changes in the cellular stress response pathways 9,10. To cope with stress, cells display several mechanisms that promote survival, including the assembly of stress granules (SGs). These are transiently formed foci that act in the triage and regulation of RNA during stress periods¹¹. Recently, SGs dysregulation has been suggested to underlie the pathogenesis of several diseases, including neurodegenerative disorders¹². SGs are dynamic structures that can have different compositions depending on the type of stress and type of cell, although RNA binding proteins (RBPs) are their main components. One of these components, which is also a core nucleator and marker of SGs is the GTPase-activating protein-binding protein 1 (G3BP1)^{13,14}. G3BP1 has an important role in mRNA stabilization, degradation, and in splicing modulation^{15,16,17}. Structurally, G3BP1 has at least two important domains, an RNA-recognition domain (RRM) and a nuclear transport factor 2-like domain (NTF2-like). The former is crucial for G3BP1 ability to bind mRNAs, whereas the latter is involved in the nuclear import of proteins through the pore complex¹⁵. Additionally, the phosphorylation of G3BP1 in its Ser-149 residue was referred to being important in SGs assembly, although recent studies do not support his hypothesis. While all these domains and catalytic site are important for the G3BP1 functions, the role of each one of them in specific steps of the RNA metabolism is yet to be elucidated^{13,14}.

Despite the extensive efforts developed over the last years, polyQ diseases pathogenesis is not completely understood, neither exist therapeutic options exist to delay or stop the disease progression. Therefore, it is essential to identify new molecular targets implicated in the disease pathogenesis and to develop new therapeutic strategies for this group of disorders.

These facts are disclosed in order to illustrate the technical problem addressed by the present disclosure.

### General Description

In the present invention it was investigated the involvement of G3BP1, a SG component in the SCA2 and SCA3 pathogenesis, and its suitability as a target for therapy. It was observed that G3BP1 overexpression led to a significant reduction in the number of cells with aggregates and in the levels of ataxin-2 and ataxin-3 proteins. The NTF2-like domain and Ser149 residue seems to be important in this mechanism of action of G3BP1. Moreover, it was found that G3BP1 levels are reduced in SCA2 and SCA3 patients' samples. Importantly, in SCA2 and SCA3 lentiviral mouse models, the knockdown of the G3bp1 levels increased the number of aggregates, highlighting an important functional role of this protein in the context of SCA2 and SCA3. On the contrary, the re-establishment of G3BP1 levels in a lentiviral mouse model of SCA2 and of SCA3 reduced neuropathological anomalies associated with the expression of mutant ataxin-2 or mutant ataxin-3, respectively. In the same line, G3BP1 expression was able to significantly reduce behaviour and neuropathological deficits in a transgenic mouse model. Altogether, the inventors had surprisingly identified G3BP1 as a relevant target for polyQ diseases, namely SCA2 and SCA3 and disclose therapeutic strategies for such diseases.

The inventors have shown that there is a reduction in G3BP1 levels in patients with a SCA2 and SCA3 disease. Based on this discovery, the inventors successfully studied the possibility to address the modulation of G3BP1 expression as a therapeutic strategy to counteract SCA2 and SCA3, by use of a vector encoding nucleic acid that expresses G3BP1 in the target cells.

It is therefore an object of the present invention to provide an isolated or artificial nucleotide sequence encoding the GTPase-activating protein-binding protein 1 (G3BP1), for use in medicine or veterinary, preferably in the treatment of polyglutamine diseases. Furthermore, the present invention is also related to a vector comprising such sequence, a host cell comprising such vector, a protein G3BP1, or a composition thereof, for use for use in medicine or veterinary, preferably in the treatment of polyglutamine diseases.

In an embodiment, the G3BP1 protein is the protein identified by the NCBI sequence reference: NP_005745.1), as encoded by the nucleotide sequence identified by the NCBI sequence reference GI: 10146 (GeneBank accession: NM_005754.3).

An aspect of the present disclosure relates to an isolated or artificial nucleotide sequence encoding the protein G3BP1, wherein the sequence is at least 95% identical to sequence selected from a list consisting of: SEQ. ID. 1; SEQ. ID. 2; SEQ. ID 3, SEQ. ID. 4; SEQ. ID 5; SEQ. ID 6; SEQ. ID 7 and mixtures thereof, for use in medicine or veterinary.

In an embodiment, the isolated or artificial nucleotide sequence for use in medicine or veterinary is identical to a sequence selected from a list consisting of: SEQ. ID. 1; SEQ. ID. 2; SEQ. ID 3, SEQ. ID; SEQ. ID 5; SEQ. ID 6; SEQ. ID 7, and mixtures thereof.

In an embodiment, the isolated or artificial nucleotide sequence may be used in the treatment of central and peripherical nervous system diseases

In another embodiment, the isolated or artificial nucleotide sequence may be used in the treatment of neurodegenerative diseases.

In another embodiment, the isolated or artificial nucleotide sequence may be used in the treatment of a movement disorder, namely lack of balance, motor coordination and/or motor performance.

In another embodiment, the isolated or artificial nucleotide sequence may be used in the treatment of a polyglutamine disease.

In another embodiment, the isolated or artificial nucleotide sequence may be used in the treatment of a polyglutamine disease wherein said diseases are positively influenced by the control of protein aggregation, wherein said control of protein aggregation is the control of protein aggregation caused by an expansion in the polyglutamine segment of the affected proteins.

In another embodiment, the isolated or artificial nucleotide sequence may be used in the treatment of a polyglutamine disease, wherein the disease is selected from the group consisting of: Huntington's disease (HD), Spinal bulbar muscular atrophy (SBMA), Dentatorubral-pallidoluysian atrophy (DRPLA), and polyglutamine repeat spinocerebellar ataxia.

In another embodiment, the isolated or artificial nucleotide sequence may be used in the treatment of a polyglutamine repeat spinocerebellar ataxia, wherein the polyglutamine repeat spinocerebellar ataxia is selected from the group consisting of: spinocerebellar ataxia type 1 (SCA1), Spinocerebellar ataxia type 2 (SCA2), Spinocerebellar ataxia type 3 (SCA3), Spinocerebellar ataxia type 6 (SCA6), Spinocerebellar ataxia type 7 (SCA7) and Spinocerebellar ataxia type 17 (SCA17).

In another embodiment, the isolated or artificial nucleotide may be to be administered directly into the brain of the patient or into the spinal cord of the patient.

In another embodiment, the isolated or artificial nucleotide may be to be administered by intravascular, intravenous, intranasal, intraventricular or intrathecal injection.

Another aspect of the present disclosure relates to a vector or construct comprising an isolated or artificial nucleotide sequence as described above.

In an embodiment, the vector is selected from the group of adenovirus, lentivirus, retrovirus, herpesvirus and Adeno-Associated Virus (AAV) vector.

In another embodiment, the vector is a lentiviral vector.

Another aspect of the present disclosure relates to a host cell comprising the vector described above for use in medicine or veterinary.

Another aspect of the present disclosure relates to a protein G3BP1 encoded by an isolated or artificial nucleotide sequence, wherein the sequence is at least 95% identical to a sequence selected from a list consisting of: SEQ. ID. 1; SEQ. ID. 2; SEQ. ID 3, SEQ. ID; SEQ. ID 5; SEQ. ID 6; SEQ. ID 7, and mixtures thereof, for use in medicine or veterinary.

Another aspect of the present disclosure relates to a pharmaceutical composition for use in medicine or veterinary comprising a therapeutically effective amount of an isolated or artificial nucleotide sequence as described above, or a vector as described above, or a host cell as described above, or a protein as described above, or combinations thereof.

Another aspect of the present disclosure relates to a kit for use in medicine or veterinary comprising an isolated or synthetic nucleotide sequence as described above, or a vector as described above, or a host cell as described above, or a protein as described above, or combinations thereof

### BRIEF DESCRIPTION OF THE DRAWINGS

The following figures provide preferred embodiments for illustrating the disclosure and should not be seen as limiting the scope of invention.

FIG. 1: Stress granules assembly mediated by sodium arsenite does not alter the number of aggregates nor protein levels of ATXN2 and ATXN3. a Confocal microscopy representative images from Neuro2a cells expressing pathological and non-pathological forms of ATXN2, fused with an eGFP tag (upper panel), treated with sodium arsenite to induce stress granules (SGs) assembly and stained with antibodies against PABP, a SGs marker (middle panel). b Representative western blot of Neuro2a lysates expressing pathological and non-pathological forms of ATXN2 and treated sodium arsenite to induce SGs assembly. Western blots were labelled using ATXN2, phospho-eIF2α and β-tubulin antibodies. c The number of cells with ATXN2 aggregates was not significantly altered upon SGs assembly (n=3 independent experiments). d The levels of non-pathological ATXN2 (ATXN2WT) protein were not significantly altered upon SGs assembly. e The levels of pathological ATXN2 (ATXN2MUT) protein were not significantly altered upon SGs assembly (n=5 independent experiments). f Confocal microscopy representative images from Neuro2a cells expressing pathological and non-pathological forms of ATXN3, fused with an eGFP tag (upper panel), treated with sodium arsenite to induce SGs assembly and stained with antibodies against PABP1. g Representative western blot of Neuro2a lysates expressing pathological and non-pathological forms of ATXN3 and treated sodium arsenite to induce SGs assembly. Western blots were labelled using ATXN3, phospho-eIF2α and β-tubulin antibodies. h The number of cells with ATXN3 aggregates was not significantly altered upon SGs assembly (n=3 independent experiments). i The levels of non-pathological ATXN3 (ATXN3WT) protein were not significantly altered upon SGs assembly. j The levels of pathological ATXN3 (ATXN3MUT) protein were not significantly altered upon SGs assembly (n=5 independent experiments). Values are expressed as mean±SEM. Scale: 10 μm.

FIG. 2: The expression of G3BP1reduces the number of cells with aggregates of ATXN2MUT and ATXN3MUT. a Confocal microscopy representative images depicting Neuroa2 cells expressing ATX2MUT in three different experimental conditions. b Confocal microscopy representative images depicting Neuroa2 cells expressing ATX3MUT in three different experimental conditions. c The number of cells with aggregates of ATX2MUT per 100 transfected cells was significantly reduced upon G3BP1 expression, compared to both control conditions. d The number of cells with aggregates of ATX3MUT per 100 transfected cells was significantly reduced upon G3BP1 expression, compared to both control conditions. (n=3 independent experiments; *P<0.05; one-way ANOVA followed by post hoc Bonferroni multiple comparison test). Values are expressed as mean±SEM. Scale: 10 μm. e Representative western blot of Neuro2a lysates expressing pathological and non-pathological forms of ATXN2, co-transfected with lacZ or with G3BP1. f Representative western blot of Neuro2a lysates expressing pathological and non-pathological forms of ATXN2, co-transfected with lacZ or with G3BP1. g The levels of ATXN2WT protein are significantly reduced upon G3BP1 expression, compared with cells control cells co-expressing lacZ. h The levels of ATXN2MUT protein are significantly reduced upon G3BP1 expression, compared with cells control cells co-expressing lacZ. i The levels of ATXN3WT protein are significantly reduced upon G3BP1 expression, compared with cells control cells co-expressing lacZ. j The levels of ATXN3MUT protein are significantly reduced upon G3BP1 expression, compared with cells control cells co-expressing lacZ. (n=5 independent experiments; *p<0.05; **p<0.01; Student's t-test).

FIG. 3: The NFT2-like domain of G3BP1 is important in the modulation of aggregation and protein levels of ATXN2MUT and ATXN3MUT. a Schematic representation of G3BP1 structural domains and the respective constructs with deleted NFT2 domain and deleted RRM domain. A: deletion. NTF2: nuclear transport factor 2 domain. Ser: serine. PxxP: proline-rich region. RRM: RNA recognition motif. RGG box: arginine and glycine rich box. b Neuro2a cells were transfected either with full length G3BP1, G3BP1-ΔRRM or G3BP1-ΔNTF. Protein lysates were analyzed through western blot depicting the expression of G3BP1 truncated forms with different molecular weight. c Confocal microscopy representative images depicting Neuroa2 cells expressing ATXN2MUT and lacZ or G3BP1 or G3BP1-ΔNTF2 or G3BP1-ΔRRM. The expression of ATXN2MUT leads to the formations of aggregates (arrowheads). d Confocal microscopy representative images depicting Neuroa2 cells expressing ATXN3MUT and lacZ or G3BP1 or G3BP1-ΔNTF2 or G3BP1-ΔRRM. The expression of ATXN3MUT leads to the formations of aggregates (arrowheads). Scale: 10 μm. e The number of cells with aggregates of ATX2MUT per 100 transfected cells with G3BP1-ΔRRM was significantly reduced compared with lacZ control condition, and increased compared to full length G3BP1 condition. The expression of G3BP1-ΔNTF2 leads to a significant increase in the number of cells with aggregates compared to all the other experimental conditions. (n=4 independent experiments; ##P<0.01 to ATXN2MUT+lacZ; ****P<0.0001 to ATXN2MUT+G3BP1; ++++P<0.0001 to ATXN2MUT+G3BP1-ΔRRM; one-way ANOVA, followed by post hoc Bonferroni multiple comparison test). f The number of cells with aggregates of ATX3MUT per 100 transfected cells with G3BP1-ΔRRM was significantly reduced compared with lacZ control condition, and increased compared to full length G3BP1 condition. The expression of G3BP1-ΔNTF2 leads to a significant increase in the number of cells with aggregates compared to ATXN3MUT+G3BP1 and ATXN3MUT+G3BP1-ΔRRM. (n=4 independent experiments; ##P<0.01 to ATXN3MUT+lacZ; ****P<0.0001 to ATXN3MUT+G3BP1; ++P<0.01 to ATXN3MUT+G3BP1-ΔRRM; one-way ANOVA, followed by post hoc Bonferroni multiple comparison test). g Representative western blot of Neuro2a lysates expressing ATXN2MUT, co-transfected with lacZ or G3BP1-ΔRRM or G3BP1-ΔNTF2. h The levels of ATXN2MUT protein are significantly reduced upon G3BP1-ΔRRM expression compared to the other experimental conditions, whereas the expression of G3BP1-ΔNTF2 leads to a significant increase in ATXN2MUT protein levels, compared to the other conditions. (n=4 independent experiments; **P<0.01 to ATXN2MUT+lacZ; ^{####}P<0.0001 to ATXN2MUT+G3BP1-ΔRRM; one-way ANOVA, followed by post hoc Bonferroni multiple comparison test). i Representative western blot of Neuro2a lysates expressing ATXN3MUT, co-transfected with lacZ or G3BP1-ΔRRM or G3BP1-ΔNTF2. j The levels of ATXN3MUT protein are significantly reduced upon G3BP1-ΔRRM expression compared to the other experimental conditions, whereas the expression of G3BP1-ΔNTF2 leads to a significant increase in ATXN3MUT protein levels, compared to ATXN3MUT+G3BP1-ΔRRM. (n=4 independent experiments; *P<0.05 to ATXN3MUT+lacZ; **P<0.01 to ATXN3MUT+lacZ; ^{###}P<0.001 to ATXN3MUT+G3BP1-ΔRRM; one-way ANOVA, followed by post hoc Bonferroni multiple comparison test). Values are expressed as mean±SEM.

FIG. 4: Ser149 phosphorylation site is important in G3BP1 action on ataxin-2 and ataxin-3 mutant proteins. a Confocal microscopy representative images depicting Neuroa2 cells expressing ATXN2MUT and wild-type G3BP1 or G3BP1(S149A) or G3BP1(S149D). In the cells expressing wild-type G3BP1 or the phosphomimetic G3BP1 (S149D) there are no aggregates of ATXN2MUT (white arrows), contrasting with the cells expressing the phospho-dead G3BP1(Ser149A), where ATXN2MUT aggregates are observed (white arrowheads). b Confocal microscopy representative images depicting Neuroa2 cells expressing ATXN3MUT and wild-type G3BP1 or G3BP1(S149A) or G3BP1(S149D). In the cells expressing wild-type G3BP1 or the phosphomimetic G3BP1 (S149D) there are no aggregates of ATXN3MUT (white arrows), contrasting with the cells expressing the phospho-dead G3BP1(Ser149A), where ATXN3MUT aggregates are observed (white arrowheads). Scale: 20 μm. c Representative western blot of Neuro2a lysates expressing ATXN2MUT, co-transfected with wild-type G3BP1 or G3BP1(S149A) or G3BP1(S149D). d Representative western blot of Neuro2a lysates expressing ATXN3MUT, co-transfected with wild-type G3BP1 or G3BP1(S149A) or G3BP1(S149D). e The levels of ATXN2MUT protein are significantly increased in cells expressing the phospho-dead G3BP1(Ser149A), compared to ATX2MUT+G3BP1 (n=3 independent experiments; *P<0.05; one-way ANOVA, followed by post hoc Bonferroni multiple comparison test). f No significant alterations were found in the ATX2MUT mRNA levels between all the experimental conditions. g The levels of ATXN3MUT protein are significantly increased in cells expressing the phospho-dead G3BP1(Ser149A), compared to the other two conditions, whereas the cells expressing the phosphomimetic G3BP1(Ser149D) are significantly reduced, compared to the other two conditions. (n=3 independent experiments; *P<0.05 to ATXN3MUT+G3BP1; ##P<0.01 to ATXN3MUT+G3BP1(Ser149A); one-way ANOVA, followed by post hoc Bonferroni multiple comparison test). Values are expressed as mean±SEM.

FIG. 5: G3BP1 mRNA and protein levels are reduced in the SCA2 and SCA3, and its silencing in the mouse brain increases aggregation. a Representative western blot for protein lysates of fibroblasts from SCA2 patients and healthy controls. b Representative western blot for protein lysates from fibroblast of SCA3 patients and healthy controls. c, d The levels of G3BP1 protein and mRNA are significantly reduced in SCA2, compared to controls. e, f The levels of G3BP1 protein and mRNA are significantly reduced in SCA3, compared to controls. (Healthy controls n=3; SCA2 n=2; SCA3 n=5). g Representative western blot for protein lysates of cerebella from a transgenic SCA3 mouse model. h, i The levels of G3BP1 protein and mRNA are significantly reduced in the SCA3 transgenic mice, compared to C57BL/6 wild-type animals (n=3-5). j Schematic representation of the injection site for the SCA2 model. Briefly, lentiviral vectors encoding ATXN2MUT and a shRNA scramble were co-injected in one hemisphere of the striatum and in the contralateral hemisphere it was co-injected ATXN2MUT and a shRNA targeting G3bp1. k At 4 weeks post-injection the animals were euthanized and brain sections labelled with ataxin-2 to highlight the presence of pathological aggregates. I The average number of ATXN2MUT aggregates is significantly increased upon shG3bp1 expression, as compared to the control hemisphere. m Schematic representation of the injection site for the SCA2 model. Briefly, lentiviral vectors encoding ATXN3MUT and a shRNA scramble 3ere co-injected in one hemisphere of the striatum and in the contralateral hemisphere it was co-injected ATXN2MUT and a shRNA targeting G3bp1. n At 4 weeks post-injection the animals were euthanized and brain sections labelled with ataxin-3 to highlight the presence of pathological aggregates. o The average number of ATXN3MUT aggregates is significantly increased upon shG3bp1 expression, as compared to the control hemisphere. (*P<0.05; **P<0.01; ***P<0.001; Student's t-test). Values are expressed as mean±SEM.

FIG. 6: G3BP1 expression reduces the number of aggregates and the loss of neuronal markers in lentiviral mouse models of SCA2 and SCA3. Mice were stereotaxically injected into the striatum either with lentiviral particles encoding for mutant forms of ATXN2 or ATXN3, or co-injected with lentiviral particles encoding for the mutant form and G3BP1. a Schematic representation of the injection site and lentiviral vectors injected in the mouse model of SCA2. Animals were bilaterally injected and euthanized 12 weeks after the injection, for tissue collection. b Schematic representation of the injection site and lentiviral vectors injected in the mouse model of SCA3. Animals were bilaterally injected and euthanized 4 weeks after the injection, for tissue collection. c Brain sections from the lentiviral mouse model of SCA2 were analysed through immunohistochemistry using ataxin-2 and DARP-32 antibodies. Images show aggregates of ATXN2MUT (black arrowheads; Scale: 20 μm) and the loss of staining (black line) of the neuronal marker DARPP-32 (Scale: 200 μm). d The hemisphere expressing G3BP1 presented a reduced number of ATXN2MUT aggregates, compared to the control hemisphere (n=5; ***p<0.0001; Student's t-test). e G3BP1 expression rescues neuronal marker loss, compared to the contralateral hemisphere only expressing ATXN2MUT (n=5; ***p<0.0001; Student's t-test). f Representative images of immunohistochemistry brain sections, from the lentiviral mouse model of SCA3. The figures show ubiquitinated ATXN3MUT aggregates (dark dots; Scale: 20 μm) and the neuronal marker DARPP-32 loss of staining (Scale: 200 μm). g The expression of G3BP1 led to a significant reduction in the number of ubiquitinated ATXN3MUT aggregates, compared to the control condition (n=7; ***p<0.0001; Student's t-test). h G3BP1 expression rescues neuronal marker loss, as compared to controls (n=7; ***p<0.0001; Student's t-test). Values are expressed as mean±SEM.

FIG. 7: Overexpression of lentiviral vectors encoding G3BP1 in the brain of wild-type mice did not produce neuronal marker loss or inflammation. Mice at 8-12 weeks of age were stereotaxically injected into the striatum (bilaterally) either with PBS or with lentiviral particles encoding for human G3BP1 and euthanized for tissue collection 4 weeks after injection. a Schematic representation of the injection site in the striatum. b Immunohistochemistry images analysis of DARPP-32 depletion volume (dashed black line; upper panel; Scale: 200 μm) and G3BP1 (bellow panel; Scale: 50 μm) labelling in brain sections from mice injected with PBS and in the contralateral hemisphere injected with lentiviral particles encoding for G3BP1. c The total area of DARPP-32 depletion, in mice brain sections, was reduced upon injection of lentiviral particles encoding for G3BP1 when compared to the injection with PBS (n=4; *P<0.05; Student's t-test). d We also labelled GFAP, a marker of astroglyosis, in brain sections from mice injected with PBS and in the contralateral hemisphere injected with lentiviral vectors encoding G3BP1. e The quantification of GFAP immunoreactivity did not detect significant differences between both hemispheres. Scale: 200 μm. Values are expressed as mean±SEM.

FIG. 8: G3BP1 expression mitigates motor deficits and neuropathological abnormalities in a SCA3 transgenic mouse model. Transgenic mice animals expressing a truncated form of the ataxin-3 protein containing 69 glutamines were stereotaxically injected in the cerebellum with lentiviral particles encoding for GFP (control group) or with G3BP1 (treated group). Mice, at 4 weeks of age, were first tested 1-2 days prior to injection and then repeatedly tested every three weeks until 9 weeks post-injection, to be euthanized at 10 weeks post-surgery. a-c Representative plots of mice motor performance at 9 weeks post injection. a Mice injected with G3BP1 significantly improved motor performance (assessed by the rotarod test), as they remain more time at the rotating rod comparing to control mice treated with GFP or non-injected mice. b Mice injected with G3BP1 significantly reduced the time needed to cross the water-filled tank and to reach the platform, comparing to control mice treated with GFP or non-injected animals. c Footprint analysis showed that mice injected with G3BP1 improved overlap measures, comparing to controls treated with GFP or non-injected animals. d Representative images of immunohistochemistry brain sections from mice cerebellum either injected with lentiviral particle encoding for GFP (control) or with G3BP1. Upper panel: ataxin-3 aggregates assessed by HA-tag immunoreactivity (arrowhead; scale: 50 μm and 200 μm). Bellow panel: Purkinje cells assessed by calbindin immunoreactivity (scale: 100 μm and 200 μm). e G3BP1 expression significantly reduced the number of HA-ataxin-3 aggregates (arrowhead), compared to control mice injected with GFP or non-injected. f G3BP1 expression significantly preserved the number of Purkinje cells (red) within lobe IX, compared to non-injected and GFP injected controls. (n=6-7; *p<0.05; one-way ANOVA followed by post hoc Bonferroni multiple comparison test). Values are expressed as mean±SEM.

FIG. 9: Sodium arsenite induces the formation of stress granules in Neuro2a cells and a reduction in overall protein synthesis. (A) Representative confocal microscopy images of Neuro2a non-treated and treated with sodium arsenite 1-hour prior fixation and depicting the SGs marker PABP immunolabelling. In the treated cells is possible to observe SGs, which are the condensate foci PABP-positive. (B) Representative western blot of protein lysates from Neuro2a cells after sunset assay. Cells were either transfected with G3BP1 or treated with sodium arsenite to induce stress granules formation. As a protein synthesis inhibition control, cells were treated with cycloheximide (CHX); membranes were probed with puromycin antibody. (C) SGs induction by sodium arsenite reduces overall protein expression. (D) Cells transfected with G3BP1 also significantly reduced overall protein synthesis compared to lacZ condition. Additionally, overall protein synthesis inhibition upon G3BP1 expression showed no differences compared to cycloheximide treatment, a known protein synthesis inhibitor (n=4; **p<0.001; ****p<0.00001; one-way ANOVA followed by post hoc Bonferroni multiple comparison test). Values are expressed as mean±SEM. Scale: 10 μm.

FIG. 10: Expression of G3BP1 and lacZ plasmids in Neuro2a cells. a Representative western blot of protein lysates from Neuro2a cells transfected with G3BP1 or lacZ, at 48 hours post-transfection. Western blots were labelled using G3BP1, β-gal and β-actin antibodies. b Representative confocal images from Neuro2a cells transfected with lacZ and immunolabeled with β-gal (white arrows), and from Neuro2a cells transfected with G3BP1 and immunolabeled G3BP1. Nuclei were stained with DAPI (blue). Scale bar: 10 μm.

FIG. 11: G3BP1 expression does not alter the levels of endogenous mouse Ataxin-2 and Ataxin-3 proteins. Neuro2A cells were co-transfected with ATXN2MUT and G3BP1 or ATXN3MUT and G3BP1. Endogenous levels of mouse Ataxin-2 and Ataxin-3 proteins were assessed. G3BP1 overexpression did not alter the endogenous levels of both (A) endogenous Ataxin-2 and (B) endogenous Ataxin-3, comparing to the control condition (n=4-5 independent experiments; Student's t-test). Values are expressed as mean±SEM.

FIG. 12: G3BP1 overexpression does not alter the expression levels of GFP. Neuro2a cells were either transfected with GFP or co-transfected with GFP and G3BP1. (A) Representative blot of Neuro2a protein lysates immunoblotted with anti-GFP antibody. (B) GFP expression was not altered upon G3BP1 expression (n=4 independent experiments; Student's t-test). Values are expressed as mean±SEM.

FIG. 13: G3BP1 co-localizes with PABP when stress granules are induced pharmacologically with sodium arsenite. Neuro2A cells were transfected with G3BP1 and treated with sodium arsenite 1-hour prior to fixation. Representative images of immunocytochemistry from Neuro2A cells depicting G3BP1 labelling. Immunolabeling of the stress granules marker protein PABP is shown in red. In Neuro2A cells transfected with G3BP1, without any treatment, co-localization with PABP was not observed. In Neuro2A cells transfected with G3BP1 and SGs assembly was induced pharmacologically with sodium arsenite treatment, PABP co-localizes with G3BP1 (yellow). Scale: 10 μm.

FIG. 14: G3BP1 assembles in PABP-positive stress granules upon sodium arsenite-induced stress. Control fibroblasts from an healthy individual, and from SCA2 and SCA3 patients were treated sodium arsenite 1 h prior to fixation. Cells were immunolabeled for G3BP1 (green) and PABP (red) and screened for co-localization of both proteins using confocal microscopy. Representative images show that G3BP1 assembles in SGs upon sodium arsenite-induced stress, highlighted by the co-localization with PABP, a marker of SGs. Nuclei were stained with DAPI (blue). Scale bar: 50 μm.

FIG. 15: Sited-directed mutagenesis of serine 149 phosphorylation site. (A) Schematic representation of G3BP1 gene structure with the mutagenesis site where a serine was changed for an alanine, creating a phospho-dead construct at the 149 aa site, G3BP1(S149A). (B) Schematic representation of G3BP1 gene structure with the mutagenesis site where a serine was changed for an aspartate, creating a phosphomimetic construct at the 149 aa site, G3BP1(S149D). (C) Representative image of G3BP1 (SEQ. ID. 1) coding region, in the proximity of the Serine-149 site. Upper histogram is showing G3BP1 sequence prior to sited-directed mutagenesis. Middle histogram is showing the G3BP1 after the site-directed mutagenesis targeted at the Serine-149->Alanine 149. The first thymine (T) nucleotide in the triplet TCT (codes for serine) was substituted by a guanine, originating the triplet GCT (codes for alanine). Below histogram is showing the G3BP1 after the site-directed mutagenesis targeted at the Serine-149->Aspartic acid 149. The thymine (T) and cytosine (C) nucleotides in the triplet TCT (codes for serine) were substituted by a guanine and adenine, originating the triplet GAT (codes for aspartate).

FIG. 16: Expression of G3BP1 reduces the levels of mutant ATXN2 and ATXN3 proteins. (A) The levels of ATXN2MUT mRNA are reduced upon expression of G3BP1, compared with the control condition (ATXN2MUT+lacZ). (B) In the same line, the levels of ATXN3MUT mRNA are reduced upon expression of G3BP1, compared with the control condition (ATXN3MUT+lacZ). (n=4 independent experiments; *P<0.05; ***P<0.001; Student's t-test). Values are expressed as mean±SEM.

FIG. 17: Immunostaining of G3BP1 is decreased in post-mortem brain samples from SCA2 patients. Representative images of immunohistochemistry in human brain samples. Post-mortem human brain biopsies from healthy individuals and SCA2 patients were immunohistologically stained for G3BP1. Upper panel: G3BP1 immunodetection from the striatum. Bellow panel: G3BP1 immunodetection from the cerebellum. Cerebellar and striatal G3BP1 staining was lost in SCA2 patients when compared with healthy individuals. Samples from two diagnosed SCA2 patients and from 3 healthy controls were analysed. Scale: 100 μm and 400 μm.

FIG. 18: Immunostaining of G3BP1 is decreased in the Purkinje cells of a SCA3 transgenic mouse model. Confocal representative images of immunohistochemistry of G3BP1 and calbindin in wild-type C57BL/6 mice and in the transgenic SCA3 mice expressing mutant ataxin-3 with 69 glutamines in the Purkinje cells of the cerebellum. In these cells is possible to observe a reduced immunostaining of G3BP1 in the transgenic animals, compared to wild-type mice. Scale: 10 μm.

FIG. 19: A shRNA targeting G3bp1, significantly reduces its levels. (A) Representative western blot from Neuro2A lysates transfect with a validated shRNA targeting mouse G3bp1 and a control shRNA scramble. (B) The shG3bp1 leads to a significant reduction in the levels of mouse endogenous G3bp1 protein, compared to control. (C) The shG3bp1 leads to a significant reduction in the levels of mouse endogenous G3bp1 mRNA, compared to control condition. (n=2-3 independent experiments; *P<0.05; **P<0.01; Student's t-test). Values are expressed as mean±SEM.

FIG. 20: G3BP1 expression in the striatum modulates the levels of ATXN3MUT in an SCA3 lentiviral mouse model. (A) Representative western blot of protein lysates from striatal punches from mice injected with lentiviral particles encoding for ATXN2MUT in one striatal hemisphere, and co-injected with lentiviral particles encoding for ATXN2MUT and G3BP1 in the contralateral hemisphere (at 4 weeks post-injection). (B) No significant alterations were found in the soluble levels of ATX2MUT between the hemispheres of the striatum. (C) The mRNA levels of ATXN2MUT were similar in both hemispheres. (D) Representative western blot of protein lysates from striatal punches from mice injected with lentiviral particles encoding for ATXN3MUT in one striatal hemisphere, and co-injected with lentiviral particles encoding for ATXN3MUT and G3BP1 in the contralateral hemisphere (at 4 weeks post-injection). (E) The levels of ATX3MUT are reduced upon G3BP1 expression, as compared with the control hemisphere. (F) The mRNA levels of ATXN3MUT were similar in both hemispheres. (SCA2 n=3 and SCA3 n=4, Student's t-test). Values are expressed as mean±SEM.

FIG. 21: Non-transduced cerebellar lobe of SCA3 transgenic mice injected with G3BP1 did not show neuropathology mitigation. SCA3 transgenic mice models expressing a truncated form of the ataxin-3 protein containing 69 glutamines were stereotaxically injected into the cerebellum with lentiviral particles encoding for G3BP1 (treated group) or injected with lentiviral particles encoding for GFP (control group). (A) Immunohistochemistry analysis of brain sections from mice cerebella either treated with lentiviral particle encoding for GFP (control) or treated with lentiviral particles encoding for G3BP1 show in the upper panel: ataxin-3 aggregates assessed by HA-tag immunoreactivity; in the bellow panel: cerebellar Purkinje cells assessed by calbindin immunoreactivity. Scale: 200 μm. Non-transduced lobe (VII) showed no differences regarding (B) the number of HA ataxin-3 aggregates and (C) the number of cerebellar Purkinje cells, comparing to the control group and to non-injected mice. (n=6 animals per group; one-way ANOVA followed by post hoc Bonferroni multiple comparison test). Values are expressed as mean±SEM.

FIG. 22: Molecular layer of the cerebellum is preserved in SCA3 transgenic mice upon injection with lentiviral particles encoding for G3BP1. SCA3 transgenic mice models expressing a truncated form of the ataxin-3 protein containing 69 glutamines were stereotaxically injected into the cerebellum with lentiviral particles encoding for GFP (control group) or injected with lentiviral particles encoding for G3BP1 (treated group). (A) Representative cresyl violet staining images of brain sections from mice cerebellum either treated with lentiviral particle encoding for GFP (control) or treated with lentiviral particles encoding for G3BP1. Upper panel: transduced lobes. Bellow panel: non-transduced lobes. Scale: 200 μm. (B) G3BP1 treatment prevented the molecular layers shrinkage (lobes II/III) of the cerebellum when compared to non-injected mice. (C) Non-transduced lobes (lobe IV/V) showed no differences regarding molecular layers thickness (n=6 animals per group; one-way ANOVA followed by post hoc Bonferroni multiple comparison test). Values are expressed as mean±SEM.

### DETAILED DESCRIPTION

The present disclosure relates to an isolated or artificial nucleotide sequence encoding the GTPase-activating protein-binding protein 1 (G3BP1), for use in medicine, preferably in the treatment of polyglutamine diseases. Furthermore, the present invention is also related to a vector comprising such sequence, a host cell comprising such vector, a protein G3BP1, or a composition thereof, for use for use in medicine, preferably in the treatment of polyglutamine diseases.

In an embodiment, an isolated or artificial sequence encoding the protein G3BP1 can be selected from the list present in Table 1.

TABLE 1
List of sequences

| Seq | Ref- | | |
|---|---|---|---|
| ID | erence | | |
| No | Name | Organism | Sequence |
| SEQ. | G3BP1 | Artificial | atggtgatggagaagcctagtcccctgctggtcgggcgggaatttgtgagacagtattacacactgctgaa |
| ID. | | | ccaggccccagacatgctgcatagattttatggaaagaactcttcttatgtccatgggggattggattcaaa |
| 1 | | | tggaaagccagcagatgcagtctacggacagaaagaaatccacaggaaagtgatgtcacaaaacttcac |
| | | | caactgccacaccaagattcgccatgttgatgctcatgccacgctaaatgatggtgtggtagtccaggtga |
| | | | tggggcttctctctaacaacaaccaggctttgaggagattcatgcaaacgtttgtccttgctcctgaggggt |
| | | | ctgttgcaaataaattctatgttcacaatgatatcttcagataccaagatgaggtctttggtgggtttgtcac |
| | | | tgagcctcaggaggagtctgaagaagaagtagaggaacctgaagaaagacagcaaacacctgaggtgg |
| | | | tacctgatgattctggaactttctatgatcaggcagttgtcagtaatgacatggaagaacatttagaggag |
| | | | cctgttgctgaaccagagcctgatcctgaaccagaaccagaacaagaacctgtatctgaaatccaagagg |
| | | | aaaagcctgagccagtattagaagaaactgcccctgaggatgctcagaagagttcttctccagcacctgc |
| | | | agacatagctcagacagtacaggaagacttgaggacattttcttgggcatctgtgaccagtaagaatcttc |
| | | | cacccagtggagctgttccagttactgggataccacctcatgttgttaaagtaccagcttcacagccccgtc |
| | | | cagagtctaagcctgaatctcagattccaccacaaagacctcagcgggatcaaagagtgcgagaacaac |
| | | | gaataaatattcctccccaaaggggacccagaccaatccgtgaggctggtgagcaaggtgacattgaac |
| | | | cccgaagaatggtgagacaccctgacagtcaccaactcttcattggcaacctgcctcatgaagtggacaa |
| | | | atcagagcttaaagatttctttcaaagttatggaaacgtggtggagttgcgcattaacagtggtgggaaat |
| | | | tacccaattttggttttgttgtgtttgatgattctgagcctgttcagaaagtccttagcaacaggcccatcatg |
| | | | ttcagaggtgaggtccgtctgaatgtcgaagagaagaagactcgagctgccagggaaggcgaccgacga |
| | | | gataatcgccttcggggacctggaggccctcgaggtgggctgggtggtggaatgagaggccctccccgtg |
| | | | gaggcatggtgcagaaaccaggatttggagtgggaagggggcttgcgccacggcagtag |
| SEQ. | G3BP1 | Artificial | gtcgggcgggaatttgtgagacagtattacacactgctgaaccaggccccagacatgctgcatagatttta |
| ID. | NTF2- | | tggaaagaactcttcttatgtccatgggggattggattcaaatggaaagccagcagatgcagtctacgga |
| 2 | like | | cagaaagaaatccacaggaaagtgatgtcacaaaacttcaccaactgccacaccaagattcgccatgttg |
| | domain | | atgctcatgccacgctaaatgatggtgtggtagtccaggtgatggggcttctctctaacaacaaccaggctt |
| | | | tgaggagattcatgcaaacgtttgtccttgctcctgaggggtctgttgcaaataaattctatgttcacaatga |
| | | | tatcttcagatac |
| SEQ. | G3BP1 | Artificial | caccaactcttcattggcaacctgcctcatgaagtggacaaatcagagcttaaagatttctttcaaagttat |
| ID. | RRM | | ggaaacgtggtggagttgcgcattaacagtggtgggaaattacccaattttggttttgttgtgtttgatgatt |
| 3 | | | ctgagcctgttcagaaagtccttagcaacaggcccatcatgttcagaggtgaggtccgtctgaatgtcgaa |
| | | | gagaagaagact |
| SEQ. | G3BP1 | Artificial | atggtgatggagaagcctagtcccctgctgcaagatgaggtctttggtgggtttgtcactgagcctcagga |
| ID. | del- | | ggagtctgaagaagaagtagaggaacctgaagaaagacagcaaacacctgaggtggtacctgatgatt |
| 4 | etion | | ctggaactttctatgatcaggcagttgtcagtaatgacatggaagaacatttagaggagcctgttgctgaa |
| | NTF2- | | ccagagcctgatcctgaaccagaaccagaacaagaacctgtatctgaaatccaagaggaaaagcctgag |
| | like | | ccagtattagaagaaactgcccctgaggatgctcagaagagttcttctccagcacctgcagacatagctca |
| | domain | | gacagtacaggaagacttgaggacattttcttgggcatctgtgaccagtaagaatcttccacccagtggag |
| | (G3BP1- | | ctgttccagttactgggataccacctcatgttgttaaagtaccagcttcacagccccgtccagagtctaagc |
| | ANTF2) | | ctgaatctcagattccaccacaaagacctcagcgggatcaaagagtgcgagaacaacgaataaatattc |
| | | | ctccccaaaggggacccagaccaatccgtgaggctggtgagcaaggtgacattgaaccccgaagaatgg |
| | | | tgagacaccctgacagtcaccaactcttcattggcaacctgcctcatgaagtggacaaatcagagcttaaa |
| | | | gatttctttcaaagttatggaaacgtggtggagttgcgcattaacagtggtgggaaattacccaattttggt |
| | | | tttgttgtgtttgatgattctgagcctgttcagaaagtccttagcaacaggcccatcatgttcagaggtgagg |
| | | | tccgtctgaatgtcgaagagaagaagactcgagctgccagggaaggcgaccgacgagataatcgccttc |
| | | | ggggacctggaggccctcgaggtgggctgggtggtggaatgagaggccctccccgtggaggcatggtgc |
| | | | agaaaccaggatttggagtgggaagggggcttgcgccacggcagtag |
| SEQ. | G3BP1 | Artificial | atggtgatggagaagcctagtcccctgctggtcgggcgggaatttgtgagacagtattacacactgctgaa |
| ID. | del- | | ccaggccccagacatgctgcatagattttatggaaagaactcttcttatgtccatgggggattggattcaaa |
| 5 | etion | | tggaaagccagcagatgcagtctacggacagaaagaaatccacaggaaagtgatgtcacaaaacttcac |
| | RRM | | caactgccacaccaagattcgccatgttgatgctcatgccacgctaaatgatggtgtggtagtccaggtga |
| | (G3BP1- | | tggggcttctctctaacaacaaccaggctttgaggagattcatgcaaacgtttgtccttgctcctgaggggt |
| | ARRM) | | ctgttgcaaataaattctatgttcacaatgatatcttcagataccaagatgaggtctttggtgggtttgtcac |
| | | | tgagcctcaggaggagtctgaagaagaagtagaggaacctgaagaaagacagcaaacacctgaggtgg |
| | | | tacctgatgattctggaactttctatgatcaggcagttgtcagtaatgacatggaagaacatttagaggag |
| | | | cctgttgctgaaccagagcctgatcctgaaccagaaccagaacaagaacctgtatctgaaatccaagagg |
| | | | aaaagcctgagccagtattagaagaaactgcccctgaggatgctcagaagagttcttctccagcacctgc |
| | | | agacatagctcagacagtacaggaagacttgaggacattttcttgggcatctgtgaccagtaagaatcttc |
| | | | cacccagtggagctgttccagttactgggataccacctcatgttgttaaagtaccagcttcacagccccgtc |
| | | | cagagtctaagcctgaatctcagattccaccacaaagacctcagcgggatcaaagagtgcgagaacaac |
| | | | gaataaatattcctccccaaaggggacccagaccaatccgtgaggctggtgagcaaggtgacattgaac |
| | | | cccgaagaatggtgagacaccctgacagtcgagctgccagggaaggcgaccgacgagataatcgccttc |
| | | | ggggacctggaggccctcgaggtgggctgggtggtggaatgagaggccctccccgtggaggcatggtgc |
| | | | agaaaccaggatttggagtgggaagggggcttgcgccacggcagtag |
| SEQ. | G3BP1 | Artificial | atggtgatggagaagcctagtcccctgctggtcgggcgggaatttgtgagacagtattacacactgctgaa |
| ID. | pospho | | ccaggccccagacatgctgcatagattttatggaaagaactcttcttatgtccatgggggattggattcaaa |
| 6 | Ser149A | | tggaaagccagcagatgcagtctacggacagaaagaaatccacaggaaagtgatgtcacaaaacttcac |
| | | | caactgccacaccaagattcgccatgttgatgctcatgccacgctaaatgatggtgtggtagtccaggtga |
| | | | tggggcttctctctaacaacaaccaggctttgaggagattcatgcaaacgtttgtccttgctcctgaggggt |
| | | | ctgttgcaaataaattctatgttcacaatgatatcttcagataccaagatgaggtctttggtgggtttgtcac |
| | | | tgagcctcaggaggaggctgaagaagaagtagaggaacctgaagaaagacagcaaacacctgaggtg |
| | | | gtacctgatgattctggaactttctatgatcaggcagttgtcagtaatgacatggaagaacatttagagga |
| | | | gcctgttgctgaaccagagcctgatcctgaaccagaaccagaacaagaacctgtatctgaaatccaagag |
| | | | gaaaagcctgagccagtattagaagaaactgcccctgaggatgctcagaagagttcttctccagcacctg |
| | | | cagacatagctcagacagtacaggaagacttgaggacattttcttgggcatctgtgaccagtaagaatctt |
| | | | ccacccagtggagctgttccagttactgggataccacctcatgttgttaaagtaccagcttcacagccccgt |
| | | | ccagagtctaagcctgaatctcagattccaccacaaagacctcagcgggatcaaagagtgcgagaacaa |
| | | | cgaataaatattcctccccaaaggggacccagaccaatccgtgaggctggtgagcaaggtgacattgaa |
| | | | ccccgaagaatggtgagacaccctgacagtcaccaactcttcattggcaacctgcctcatgaagtggaca |
| | | | aatcagagcttaaagatttctttcaaagttatggaaacgtggtggagttgcgcattaacagtggtgggaaa |
| | | | ttacccaattttggttttgttgtgtttgatgattctgagcctgttcagaaagtccttagcaacaggcccatcat |
| | | | gttcagaggtgaggtccgtctgaatgtcgaagagaagaagactcgagctgccagggaaggcgaccgacg |
| | | | agataatcgccttcggggacctggaggccctcgaggtgggctgggtggtggaatgagaggccctccccgt |
| | | | ggaggcatggtgcagaaaccaggatttggagtgggaagggggcttgcgccacggcagtag |
| SEQ. | G3BP1 | Artificial | atggtgatggagaagcctagtcccctgctggtcgggcgggaatttgtgagacagtattacacactgctgaa |
| ID. | pospho | | ccaggccccagacatgctgcatagattttatggaaagaactcttcttatgtccatgggggattggattcaaa |
| 7 | Ser149D | | tggaaagccagcagatgcagtctacggacagaaagaaatccacaggaaagtgatgtcacaaaacttcac |
| | | | caactgccacaccaagattcgccatgttgatgctcatgccacgctaaatgatggtgtggtagtccaggtga |
| | | | tggggcttctctctaacaacaaccaggctttgaggagattcatgcaaacgtttgtccttgctcctgaggggt |
| | | | ctgttgcaaataaattctatgttcacaatgatatcttcagataccaagatgaggtctttggtgggtttgtcac |
| | | | tgagcctcaggaggaggatgaagaagaagtagaggaacctgaagaaagacagcaaacacctgaggtg |
| | | | gtacctgatgattctggaactttctatgatcaggcagttgtcagtaatgacatggaagaacatttagagga |
| | | | gcctgttgctgaaccagagcctgatcctgaaccagaaccagaacaagaacctgtatctgaaatccaagag |
| | | | gaaaagcctgagccagtattagaagaaactgcccctgaggatgctcagaagagttcttctccagcacctg |
| | | | cagacatagctcagacagtacaggaagacttgaggacattttcttgggcatctgtgaccagtaagaatctt |
| | | | ccacccagtggagctgttccagttactgggataccacctcatgttgttaaagtaccagcttcacagccccgt |
| | | | ccagagtctaagcctgaatctcagattccaccacaaagacctcagcgggatcaaagagtgcgagaacaa |
| | | | cgaataaatattcctccccaaaggggacccagaccaatccgtgaggctggtgagcaaggtgacattgaa |
| | | | ccccgaagaatggtgagacaccctgacagtcaccaactcttcattggcaacctgcctcatgaagtggaca |
| | | | aatcagagcttaaagatttctttcaaagttatggaaacgtggtggagttgcgcattaacagtggtgggaaa |
| | | | ttacccaattttggttttgttgtgtttgatgattctgagcctgttcagaaagtccttagcaacaggcccatcat |
| | | | gttcagaggtgaggtccgtctgaatgtcgaagagaagaagactcgagctgccagggaaggcgaccgacg |
| | | | agataatcgccttcggggacctggaggccctcgaggtgggctgggtggtggaatgagaggccctccccgt |
| | | | ggaggcatggtgcagaaaccaggatttggagtgggaagggggcttgcgccacggcagtag |

In order that the present invention may be more readily understood, certain terms are first defined. In addition, it should be noted that whenever a value or range of values of a parameter are recited, it is intended that values and ranges intermediate to the recited values are also intended to be part of this invention. The articles “a” and “an” are used herein to refer to one or to more than one (i.e., to at least one) of the grammatical object of the article. By way of example, “an element” means one element or more than one element, e.g., a plurality of elements. The term “including” is used herein to mean, and is used interchangeably with, the phrase “including but not limited to”. The term “or” is used herein to mean, and is used interchangeably with, the term “and/or,” unless context clearly indicates otherwise. For example, “sense strand or antisense strand” is understood as “sense strand or antisense strand or sense strand and antisense strand.” The term “about” is used herein to mean within the typical ranges of tolerances in the art. For example, “about” can be understood as about 2 standard deviations from the mean. In certain embodiments, about means+10%. In certain embodiments, about means+5%. When about is present before a series of numbers or a range, it is understood that “about” can modify each of the numbers in the series or range. The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 15 nucleotides of a 21 nucleotide nucleic acid molecule” means that 15, 16, 17, 18, 19, 20, or 21 nucleotides have the indicated property. When at least is present before a series of numbers or a range, it is understood that “at least” can modify each of the numbers in the series or range.

In the context of the invention, the terms “treatment”, “treat” or “treating” are used herein to characterize a therapeutic method or process that is aimed at (1) slowing down or stopping the progression, aggravation, or deterioration of the symptoms of the disease state or condition to which such term applies; (2) alleviating or bringing about ameliorations of the symptoms of the disease state or condition to which such term applies; and/or (3) reversing or curing the disease state or condition to which such term applies.

As used herein, the term “subject” or “patient” refers to an animal, preferably to a mammal, even more preferably to a human, including adult and child. However, the term “subject” can also refer to non-human animals, in particular mammals such as mouse, and non-human primates.

As used herein, the term “gene” refers to a polynucleotide containing at least one open reading frame that is capable of encoding a particular polypeptide or protein after being transcribed or translated.

As used herein, the terms “coding sequence” or “a sequence which encodes a particular protein”, denotes a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

“G, “C”, “A,” “T,” and “U” each generally stand for a nucleotide that contains guanine, cytosine, adenine, thymidine, and uracil as a base, respectively.

In an embodiment, the invention describes an isolated or artificial sequence or a variant thereof for use in medicine.

The variants include, for instance, naturally-occurring variants due to allelic variations between individuals (e.g., polymorphisms), alternative splicing forms, etc. The term variant also includes G3BP1 gene sequences from other sources or organisms. Variants are preferably substantially homologous to one of the sequences SEQ. ID. 1-7, i.e., exhibit a nucleotide sequence identity of typically at least 90%, preferably at least 95%, more preferably at least 98%, more preferably at least 99% with one of the sequences SEQ. ID. 1-7.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (over the whole the sequence) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. The sequence identity values, which are indicated in the present subject matter as a percentage were determined over the entire amino acid sequence, using BLAST with the default parameters.

In an embodiment, the vector use according to the present invention is a non-viral vector. Typically, the non-viral vector may be a plasmid encoding G3BP1. This plasmid can be administered directly or through a liposome, an exosome or a nanoparticle.

### Viral Vectors

Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins which mediate cell transduction.

The terms “gene transfer” or “gene delivery” refer to methods or systems for reliably inserting foreign DNA into host cells. Such methods can result in transient expression of non-integrated transferred DNA, extrachromosomal replication and expression of transferred replicons (e.g. episomes), or integration of transferred genetic material into the genomic DNA of host cells.

In an embodiment, examples of viral vector include adenovirus, lentivirus, retrovirus, herpes-virus and Adeno-Associated virus (AAV) vectors.

Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478.

In a preferred embodiment, lentiviral vectors are employed.

Lentiviral vectors typically are generated by trans-complementation in packaging cells that are co-transfected with a plasmid containing the vector genome and the packaging constructs that encode only the proteins essential for lentiviral assembly and function. A self-inactivating (SIN) lentiviral vector can be generated by abolishing the intrinsic promo ter/enhancer activity of the HIV-1 LTR, which reduces the likelihood of aberrant expression of cellular coding sequences located adjacent to the vector integration site (see, e.g., Vigna et al., J. Gene Med., 2: 308-316 (2000); Naldini et al., Science, 272: 263-267 (1996); and Matrai et al., Molecular Therapy, 18(3): 477-490 (2010)). The most common procedure to generate lentiviral vectors is to co-transfect cell lines (e.g., 293T human embryonic kidney cells) with a lentiviral vector plasmid and three packaging constructs encoding the viral Gag-Pol, Rev-Tat, and envelope (Env) proteins.

### Delivery of Vectors

Methods of delivery, or administration, of viral vectors to neurons and/or astrocytes and/or oligodendrocytes and/or microglia include generally any method suitable for delivery vectors to said cells, directly or through hematopoietic cells transduction, such that at least a portion of cells of a selected synaptically connected cell population is transduced. The vector may be delivered to any cells of the central nervous system, cells of the peripheral nervous system, or both. Preferably, the vector is delivered to cells of the brain. Generally, the vector is delivered to the cells of the brain, including for example cells of brainstem (medulla, pons, and midbrain), cerebellum, susbtantia nigra, striatum (caudate nucleus and putamen), frontotemporal lobes, visual cortex, spinal cord or combinations thereof, or preferably any suitable subpopulation thereof.

Additional routes of administration may also comprise local application of the vector under direct visualization, e. g., superficial cortical application, intranasal application, or other nonstereotactic application.

The target cells of the vectors of the present invention are cells of the brain of a subject afflicted with PolyQ SCA, preferably neural cells. Preferably the subject is a human being, generally an adult but may be a child or an infant.

The present invention also encompasses delivering the vector to biological models of the disease. In that case, the biological model may be any mammal at any stage of development at the time of delivery, e. g., embryonic, foetal, infantile, juvenile or adult, preferably it is an adult. Furthermore, the target cells may be essentially from any source, especially nonhuman primates and mammals of the orders Rodenta (mice, rats, rabbit, hamsters), Carnivora (cats, dogs), and Arteriodactyla (cows, pigs, sheep, goats, horses) as well as any other non-human system (e. g. zebrafish model system).

Preferably, the method of the invention comprises intracerebral administration, through stereotaxic injections. However, other known delivery methods may also be adapted in accordance with the invention. For example, for a more widespread distribution of the vector across the brain, it may be injected into the cerebrospinal fluid, e. g., by lumbar puncture, cisterna magna or ventricular puncture. To direct the vector to the brain, it may be injected into the spinal cord or into the peripheral ganglia, or the flesh (subcutaneously or intramuscularly) of the body part of interest. In certain situations the vector can be administered via an intravascular approach. For example, the vector can be administered intra-arterially (carotid) in situations where the blood-brain barrier is disturbed. Moreover, for more global delivery, the vector can be administered during the “opening” of the blood-brain barrier achieved by infusion of hypertonic solutions including mannitol or ultra-sound local delivery.

The vectors used herein may be formulated in any suitable vehicle for delivery. For instance, they may be placed into a pharmaceutically acceptable suspension, solution or emulsion. Suitable mediums include saline and liposomal preparations. More specifically, pharmaceutically acceptable carriers may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Intravenous vehicles include fluid and nutrient replenishers, electrolyte replenishers (such as those based on Ringer's dextrose), and the like.

Preservatives and other additives may also be present such as, for example, antimicrobials, antioxidants, chelating agents, and inert gases and the like.

A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposomes or exosomes.

The invention will be further illustrated by the following example. However, this example and the accompanying figures should not be interpreted in any way as limiting the scope of the present invention.

### Example

### Material and Methods

### Plasmid Vectors

In an embodiment, plasmids encoding for human ataxin-3 contain 28 glutamines (pEGFP-C1-Ataxin3Q28; #22122; Addgene) or 84 glutamines (pEGFP-C1-Ataxin3Q84; #22123; Addgene) were a gift from Henry Paulson and both are fused with a GFP protein at the N-terminal¹⁸. Plasmids encoding for human ataxin-2 containing 22 glutamines (pEGFP-Ataxin2Q22) or 104 glutamines (pEGFP-Ataxin2Q104) were kindly provided by Prof. Stefan Pulst¹⁹. The LacZ gene was cloned in our laboratory under the control of a phosphoglycerate kinase promoter (PGK)²⁰, and the GFP construct was cloned as previously described²¹. The plasmid encoding for human G3BP1 (SEQ. ID. 1) purchased from Source Bioscience, was cloned into a lentiviral vector backbone using the Gateway™ LR Clonase™ II Enzyme Mix, Invitrogen, according to the manufacturer instructions. The G3BP1-ΔNTF2 (G3BP1 deleted at the site 11-133) and G3BP1-ΔRRM (G3BP1 deleted at the site 340-415) constructs were synthesized from GeneScript and cloned into the vector pcDNA3.1+N-MYC. A validated shRNA targeting mouse G3bp1 (#MSH031039-LVRU6MP-b) and a shRNA scramble, as control (with no known target, #CSHCTR001-LVRU6MP) were acquired from GeneCopoeia (USA).

### Lentiviral Vector Comprising the Plasmid Encoding for Human G3BP1

In an embodiment, the plasmid encoding for human G3BP1 (one of the sequences SEQ. ID. 1 to 7) were cloned into a self-inactivating lentiviral vector under the control of PGK promoter using the Gateway™ LR Clonase™ II Enzyme Mix, Invitrogen, according to the manufacturer instructions. The lentiviral vectors were produced in HEK (human embryonic kidney) 293T cells using a four-plasmid system described previously²⁵. The viral productions were quantified using a RetroTek HIV-1 p24 Antigen Enzyme-Linked Immunoabsorbent Assay (ELISA) (ZeptoMetrix), according to manufacturer's indications.

### G3BP1 Mutagenesis of Serine149 Residue

In an embodiment, sited-directed mutagenesis was performed using NZY Mutagenesis kit (NZYTech) according to manufacturer's indications. In the human variant of G3BP1 (GeneBank accession: DQ893058.2), a serine was changed by an alanine or an aspartate at the site 149 to generate a G3BP1 phospho-dead mutant (G3BP1_S149A) or a G3BP1-phosphomimic mutant (G3BP1_S149D), respectively. The pair of primers used to induce the substitution S149A were: SEQ. ID. 8: 5′-CT GAG CCT CAG GAG GAG GCT GAA GAA GAA GTA GAG-3′ and SEQ. ID. 9: 5′-CT CTA CTT CTT CTT CAG CCT CCT CCT GAG GCT CAG-3′. The pair of primes used to induce the substitution S149D were: SEQ. ID. 10: 5′-CT GAG CCT CAG GAG GAG GAT GAA GAA GAA GTA GAG-3′ and SEQ. ID. 11: 5′-CTC TAC TTC TTC TTC ATC CTC CTC CTG AGG CTC AG-3′. The mutations S149A and S149D were confirmed by DNA sequencing (Eurofins Genomics).

### Neuroblastoma Culture and Transfection

In an embodiment, mouse neuroblastoma cell line (Neuro2a cells) acquired from the American Type Culture Collection cell biology bank (CCL-131) were cultured in Dulbecco's modified Eagle medium (DMEM), supplemented with 10% (v/v) foetal bovine serum (FBS), 100 U/mL penicillin and 100 μg/mL streptomycin. Cells were seeded onto 12- or 6-multiwell plates. After 24 h of growth, cells were transfected using polyethylenimine reagent (PEI; PEI MAX Polysciences, Inc.) following the manufacturer's instructions, with a concentration of 0.5-1 μg of DNA per well. For SGs induction experiments, cells were treated with sodium arsenite (SA, Sigma Aldrich 10 μg/mL) to a final concentration of 0.05 M, 1 h before harvest.

### Human Fibroblast Culture

In an embodiment, patients fibroblasts from SCA2, SCA3, and healthy individuals were obtained from Coriell Institute or kindly provided by collaborators²², being fully characterized for CAG expansions: SCA2 (patient 1: 22/41; patient 2: 20/44); SCA3 (patient 1: 18/79; patient 2: 22/77; patient 3: 23/80; patient 4: 23/71; patient 5: 24/74); healthy controls (1: 14/19; 2: 14/23; 3: 22/23; 4: 22/23). Fibroblast cells were kept in culture in Dulbecco's modified Eagle medium (DMEM), supplemented with 15% (v/v) foetal bovine serum (FBS), 100 U/mL penicillin and 100 μg/mL streptomycin. All cell cultures were maintained at 37° C. in a humidified atmosphere containing 5% CO2.

### Translation Rate Assay (SUnSET Protocol)

In an embodiment, it was used a method that allows the monitoring and quantification of global protein synthesis based on the incorporation of puromycin during translation. N2a cells were plated into multiwell plates and transfected with lacZ or G3BP1. Twenty-four hours post-transfection the cells were incubated with 10 mg/ml of puromycin (Sigma) for 15 min, and after collected for western blot processing. As a positive control for the translation inhibition, some cells were incubated with 10 mM of cycloheximide (CHX, Sigma) for 15 min, and then incubated with 10 mg/ml of puromycin (Sigma) for an additional 15 min. For the stress granules condition, the cells were treated for 1 h with 0.05M sodium arsenite and then incubated with 10 mg/ml of puromycin (Sigma) for an additional 15 min. Additional controls of non-treated cells were also used.

### Human Brain Tissue

In an embodiment, post-mortem striatum and cerebellum brain tissue from clinically and genetically confirmed SCA2 patients were obtained from the NIH NeuroBioBank (USA). Control striatum and cerebellum tissues from healthy individuals, without neurological conditions diagnosed were obtained from NIH NeuroBioBank (USA). Tissues preserved in 4% PFA solution, were dehydrated in a 30% sucrose/PBS for 48 h, cryoprotected at −80° C. degrees, dissected in 40 μm slices using a cryostat (Cryostar NX50, ThermoFisher Scientific) and stored in free floating PBS/sodium azide solution at 4° C.

### Animals

In an embodiment, adult C57BL/6 J wild-type animals, and transgenic SCA3 mice²³, breed in in the animal facility of the Universidade do Algarve, were used. The animals were maintained in a temperature-controlled room on a 12 h light-12 h fark cycle. Food and water were dispensed ad libitum. All experiments were carried out according to the European Community Council directive (86/609/EEC) for the care and use of laboratory animals. The researchers received certified training (FELASA course) and approval to perform the experiments from the Portuguese authorities (Direcção Geral de Alimentação e Veterinária) in the project Neuropath (421/2019).

### Lentiviral Vectors

In an embodiment, the cDNA encoding for human G3BP1, GFP, ATXN2MUT, and for ATXN3MUT was cloned in a self-inactivating lentiviral vector under the control of PGK promoter, as described previously²⁴. The lentiviral vectors were produced in HEK (human embryonic kidney) 293T cells using a four-plasmid system described previously²⁵. The viral productions were quantified using a RetroTek HIV-1 p24 Antigen Enzyme-Linked Immunoabsorbent Assay (ELISA) (ZeptoMetrix), according to manufacturer's indications.

### In Vivo Injection of Lentiviral Vectors

In an embodiment, for the stereotaxic injection of lentiviral vectors, concentrated viral stocks were thawed on ice and homogenized. The animals were anesthetized through intraperitoneal injection (IP) of a mixture of ketamine (75 mg/kg, Nimatek, Dechra) with medetomidine (0.75 mg/kg, DOMTOR®, Esteve). For the SCA2 lentiviral mouse model, mice (10-12 weeks old) were injected with lentiviral particles encoding for human ATXN2MUT containing 82 glutamines or encoding for ATXN2MUT and G3BP1 at the left and right hemispheres of striatum, respectively, according to the following brain coordinates relative to bregma: Antero-Posterior (+0.6), Medial-Lateral (+/−1.8), Dorsal-Ventral (−3.3)²⁶. A concentration of 400 ng p24/μl of lentivirus were injected at a rate of 0.20 μl/min. For the SCA3 lentiviral mice, viral particles encoding for human ATXN3MUT containing 72 glutamines or encoding for ATXN3MUT and G3BP1, were injected into mouse striatum (left hemisphere and right hemisphere, respectively) at 400 ng of p24/ml, using the same coordinates described above. To perform safety assays, wild-type C57/BL6 mice (10-12 weeks old) were injected into the striatum with lentiviral particles encoding for G3BP1 at a concentration of 400 ng p24/μl of lentivirus, using the same coordinates described above. For the transgenic animals, lentiviral particles encoding for G3BP1 or GFP, as respective control, were injected into mice cerebella (4 weeks old), at a concentration of 800 ng p24/μl of lentivirus at the coordinates: −1.6 mm rostral to lambda, 0.0 mm midline, and −1.0 mm ventral to the skull surface, with the mouth bar set at −3.321. For the G3bp1 silencing studies in SCA2, wild-type C57/BL6 mice (10-12 weeks old) were injected in the striatum with lentiviral particles encoding for human ATXN2MUT containing 82 glutamines and with lentiviral particles encoding for an shRNA scramble, whereas in the contralateral hemisphere the animals were injected with lentiviral particles encoding for human ATXN2MUT containing 82 glutamines and with lentiviral particles encoding for an shRNA targeting mouse G3bp1. For SCA3, the procedure was similar but using lentiviral particles encoding for human ATXN3MUT containing 72 glutamines. The lentiviral particles were injected at a concentration of 400 ng p24/μl of lentivirus, using the same coordinates described above. All stereotaxic injections were performed by means of an automatic injector (Stoelting Co.) using a 34-gauge blunt-tip needle linked to a Hamilton syringe. Mice were sacrificed for posterior analysis, a few weeks after surgery, according to the model, SCA2 lentiviral mice: 4 weeks and 12 weeks; SCA3 lentiviral mice: 4 weeks; G3BP1 injected mice: 4 weeks; SCA3 transgenic mice: 9 weeks.

### Behavioural Testing

In an embodiment, the transgenic mice were subjected to several motor behaviour tests starting before the stereotaxic injection (4 weeks of age), every 3 weeks until 9 weeks post-injection. Motor and gait coordination were accessed by rotarod and footprint tests in a blind fashion way following the same procedure described before²¹. In the footprint test analysis, steps taken by mice at the beginning and at the end of the walking test are not included and not considered for the measures. Swimming performance was assessed by placing mice at one end of a rectangular tank (100×10.5×20 cm), filled with water at room temperature. Mice freely swam for 1 m until they reached a platform and the time taken to transverse the tank was recorded. Mice performed the trial three times, with an interval of 15-20 minutes per trial. The mean of the time taken to cross the tank in the tree trials was used for statistical analysis.

### Tissue Processing

In an embodiment, animals were sacrificed by sodium pentobarbital overdose and either transcardially perfused with 0.1M phosphate buffer solution and a 4% paraformaldehyde fixative solution (Sigma Aldrich) for immunohistochemical assays or had cervical dislocation and striatal punches of the brains, using a Harris Core pen with 2.5 mm diameter (Ted Pella Inc.), for qPCR and western blot analysis. The brains and the striatal punches collected were post-fixed in 4% paraformaldehyde for 24 h, dehydrated in a 30% sucrose/0.1M phosphate buffer solution (PBS) for 48 h and cryoprotected at −80° C. Sagittal or coronal brain sections of 30 μm and 25 μm, respectively, were obtained using the cryostat-microtome model CryoStar NX50 (Thermofisher). For preservation, brain sections were stored at 4° C., free-floating in 0.02% (w/v) sodium azide PBS

### Cresyl Violet Staining

In an embodiment, to stain brain sections with cresyl violet, they were mounted in gelatin-coated microscope slides. Brain sections were sequentially submersed in water, ethanol 96% (v/v), ethanol 100% (v/v), xylene, ethanol 75% (v/v) and the 0.1% (w/v) cresyl violet solution. To wash slices, brain sections were sequentially submersed in water, ethanol 75% (v/v), ethanol 96% (v/v), ethanol 100% (v/v) and xylene. Finally, brain sections were mounted with Eukitt (Sigma-Aldrich). Images were acquired with 10× objective in a Zeiss Axio Imager Z2.

### Immunocytochemistry

In an embodiment, for the immunocytochemical procedure, cells were fixed using 4% paraformaldehyde (PFA) fixative solution for 20 min and washed with 0.1 M phosphate buffer solution (PBS). Samples were then incubated in PBS containing 0.1% Triton™ X-100 for 10 min. Blocking in PSB with 1% of bovine serum albumin (Sigma) was performed for 30 min. Samples were incubated with the primary antibody overnight in the proper dilution at 4° C. and with the secondary antibody (1:200) for 2 h at room temperature. The secondary antibody was coupled to a fluorophore (Alexa Fluor®, Invitrogen). Finally, the coverslips were mounted on microscope slides using Fluoromount-G mounting media with DAPI (Invitrogen).

### Immunohistochemistry

In an embodiment, the immunohistochemical procedure, for light imaging, started with the incubation of brain sections in phenylhydrazine diluted in phosphate buffer solution (1:1000; 15 min, 37° C.). For the human brain sections an additional step with a Tris-buffered saline pH 9 antigen retrieval method (30 min, 95° C.) was performed. Brain sections went through blocking in 10% normal goat serum in 0.1% Triton™ X phosphate-buffered solution (1 h, room temperature) and incubation with the respective primary (overnight at 4° C.) and secondary biotinylated antibodies (2 h at room-temperature) diluted in blocking solution, followed a reaction with the Vectastain elite avidin-biotin-peroxidase kit and by 3,3′-diaminobenzidine substrate (both from Vector Laboratories). Then, the sections were assembled over microscope slides, dehydrated in increasing degree ethanol solutions (75, 96 and 100%) and xylene, and finally cover slipped using mounting medium Eukitt (O. Kindler GmbH & CO). For fluorescence immunohistochemistry procedures, brain sections were incubated in the above-described blocking solution, followed by primary and secondary antibodies incubation. Brain sections were mounted in microscope slides with Fluoromount-G Mounting medium with 41, 6-Diamidino-2-Phenylindole, (DAPI) (Invitrogen).

### Immunochemical Antibodies

In an embodiment, for immunochemical procedures, the following primary antibodies were used: mouse anti-ataxin-2 (1:1000, ref. 611378, BD Biosciences); mouse anti-ubiquitin (1:1000, ref. 3936S, Cell Signaling) rabbit anti-DARPP-32 (1:1000, ref. AB10518, Merck Millipore); rabbit anti-G3BP1 (1:1000, ref. 07-1801, Millipore); mouse anti-human G3BP1 (1:1000, ref. 611126, BD Biosciences); anti-G3BP1 (1:1000, ref. 05-1938; Sigma-Aldrich); mouse anti-GFAP (1:1000, ref. 644702, BioLegend); rabbit anti-HA (1:1000, ref. Ab9110, Abcam); mouse anti-calbindin D-28K (1:1000, ref. C9848, Sigma Aldrich); mouse anti-PABP-1 (1:1000, ref. 04-1467, Millipore); mouse β-Gal (14B7) (1:500, ref. 2372, Cell Signaling Technology).

### Image Quantitative Analysis and Data Processing

In an embodiment, immunocytochemistry images were acquired in a Zeiss Axio Imager Z2 for quantification and in a Zeiss LSM710 confocal microscope for representative images. Quantitative analysis was blindly performed by counting the number of cells with aggregates within 100 transfected cells, using the 40x or 63x objective for each condition in each independent experiment. Immunohistochemistry images from the lentiviral mouse models were acquired with 20× objective in a Zeiss Axio Imager Z2 and Axio Scan.Z1 Slide Scanner microscopes. For quantification of ataxin-2 aggregates and DARP-32 staining loss, 18 coronal sections per animal were analyzed in ZEN lite software (Zeiss), so that a complete rostrocaudal picture of the striatum was obtained. Ataxin-2 inclusions were manually counted in all animals. DARP-32 neuronal lesion area was manually measured for all animals, allowing quantification of depleted volume according to the formula: volume=d*(a1+a2+a3), where d is the distance between serial sections (200 μm) and a1+a2+a3 are depleted areas for each individual section. Immunohistochemistry images from the transgenic mouse animals were acquired 8 sagittal sections, spanning 280 μm between them, of the entire cerebellum, stained with anti-HA, anti-Calbindin and DAPI were acquired with a Zeiss Axio Imager Z2 microscope using a 20x objective. For each section, the number of cells with HA aggregates and Purkinje cells were blindly counted in all cerebellar lobules using an image analysis software (ZEN 2.1 lite, Zeiss).

### Western Blot

In an embodiment, samples were either lysed in 10x RIPA solution (Merck Millipore) if cell extracts or homogenized in a urea/DTT solution if mouse striatal punches, both containing a cocktail of protease inhibitors (Roche), followed by an ultrasound sonication of 30 sec ON, 30 sec OFF, 5 cycles (Bioruptor Pico). Protein concentration levels were determined using Pierce™ BCA Protein Assay Kit (Thermo Scientific) for cell lysates and NZYBradford reagent (Nzytech) for mouse samples. Protein extracts were resolved in SDS-polyacrylamide gels (7.5% and 12%), followed by protein transfer to PVDF membrane (Merck Millipore), membrane blocking in TBS-T with 3% of BSA or 5% of milk, and antibody probing overnight at 4° C. for primary and 2 h at room temperature for secondary. The following antibodies were used: mouse anti-ataxin-2 (1:1000, ref. 611378, BD Biosciences); mouse anti-ataxin-3(1H9) (1:1000, ref. MAB5360, BD Biosciences); rabbit anti-G3BP1 (1:1000, ref. 07-1801, Millipore); mouse anti-human G3BP1 (1:1000, ref. 611126, BD Biosciences); anti-G3BP1 (1:1000, ref. 05-1938; Sigma-Aldrich); mouse anti-β-actin (1:5000, ref. A5316, Sigma Aldrich) mouse anti-β-tubulin (1:5000, ref. T7816, Sigma); mouse anti-puromycin (1:250, ref. MABE343, Millipore); mouse anti-GFP (1:1000, ref. 668205, BioLegend); mouse β-Gal (14B7) (1:500, ref. 2372, Cell Signaling Technology). Membranes were resolved using Enhanced Chemiluminescence (GE Healthcare) and scanned with a ChemiDoc™ XRS+ (Bio-Rad). Optical densiometric analysis was carried out using Image J software.

### RT-qPCR

In an embodiment, total RNA from mouse striatal punches started by Trizol (Invitrogen) tissue dissociation and RNA/DNA/protein chloroform separation. Then, both mouse and cell samples were extracted with NZY Total RNA Isolation kit (Nzytech). RNA concentration and purity were determined using a NanoDrop 2000 spectrophotometer (Thermo Fisher Scientific). cDNA molecules of 1 μg of RNA were produced using iScript cDNA synthesis kit (Bio-Rad) according to manufacturer recommendations. Quantitative RT-qPCR was performed with the SsoAdvanced™ Universal SYBR® Green Supermix (Bio-Rad), using home-made primers for gene of interest and for the human GAPDH housekeeping gene as a control and performed in a CFX96 Touch Real-Time PCR Detection System (Bio-Rad). mRNA expression levels relative to mRNA gene control were determined using amplification values. The following primers were used: human ATXN2 (QT01852480) and human ATXN3 (QT00094927) from QuantiTect Primer Assays, Qiagen. Human G3BP1 (Forward SEQ. ID. 12: 5′-GAA ATC CAA GAG GAA AAG CC-3′; Reverse SEQ. ID. 13: 5′-CCC AAG AAA ATG TCC TCA AG), human GAPDH (Forward SEQ. ID. 14: 5′-ACA GTT GCC ATG TAG ACC-3′; Reverse SEQ. ID. 15: 5′-TTG AGC ACA GGG TAC TTT A-3′) and mouse Hprt (Forward SEQ. ID. 16: 5′-AGG GAT TTG AAT CAC GTT TG-3′; Reverse SEQ. ID. 17: 5′-TTT ACT GGC AAC ATC AAC AG-3′) from KiCqStart Pre-designed Primers, Sigma-Aldrich.

### Statistical Analysis

In an embodiment, statistical analysis was performed using either Student's t-test or one-way ANOVA complemented with Bonferroni multiple comparisons test, resorting to GraphPad software (La Jolla).

### Results

Stress Granules Assembly does not Alter the Levels of ATXN2 and ATXN3 Proteins.

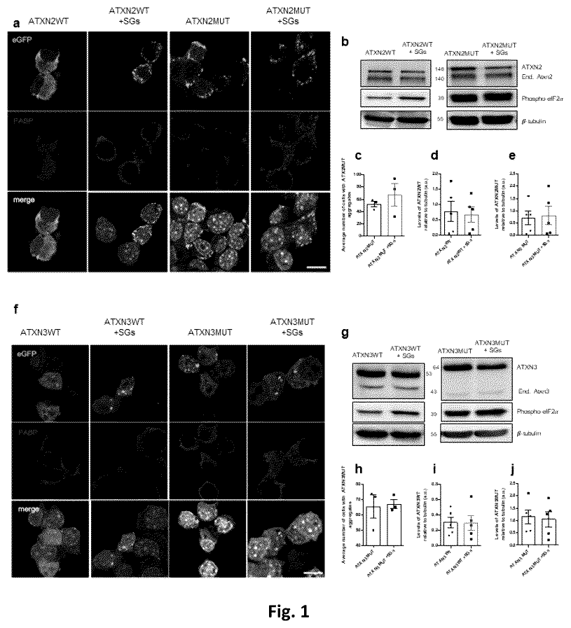

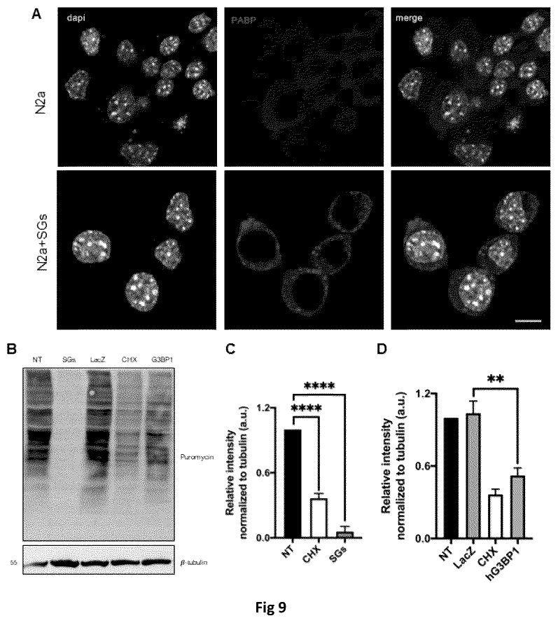

SGs are cellular foci formed in response to stress in which mRNAs, translation factors, and RBPs coalesce together to prevent cellular damage^{27,28}. Therefore, the inventors of the present disclosure investigated the impact of SGs assembly in ATXN2 and ATXN3 proteins dynamics, both in pathological (ATXN2MUT and ATXN3MUT) and non-pathological forms (ATXN2WT and ATXN3WT). For that, in Neuro2a cells expressing ATXN2 (ATXN2WT: pEGFP-ATXN2-Q22 or ATXN2MUT: pEGFP-ATXN2-Q104) or ATXN3 (ATXN3WT: pEGFP-ATXN3-Q24 or ATXN3MUT: pEGFP-ATXN3-Q844), SGs assembly was pharmacologically induced using sodium arsenite (FIG. 9a). As previously reported, ataxin-2 is recruited to SGs²⁹, however, ataxin-3 is not. The expression of the mutant forms of both proteins leads to the formation of aggregates (FIG. 1a, 1f). However, the assembly of SGs did not alter the number of cells with ATXN2MUT or ATXN3MUT aggregates, compared to the control conditions (ATXN2MUT and ATXN3MUT, respectively), in which stress stimulus was not induced (FIG. 1c, 1h). The non-pathological forms of the proteins do not form aggregates, however when SGs assembly is induced there is the formation of aggregates-like structures in both ATXN2WT and ATXN3WT conditions (FIG. 1a, 1f). SGs assembly is accompanied by the phosphorylation of eiF2α, and translation inhibition³⁰, leading to a reduction in the overall protein synthesis (FIG. 9b, c). Thus, it was then investigated if the levels of ATXN2 and ATXN3 proteins are altered upon SGs assembly and therefore their levels were analysed by western blot (FIG. 1b, 1g). In line with the results observed on the aggregates, it was not observed alterations in the levels of these proteins among the different experimental conditions, neither in the non-pathological (FIG. 1d, 1i) nor in the pathological (FIG. 1e, 1j) protein forms. Altogether, these results showed that although SGs assembly reduces the overall translation, it does not seem to interfere with ATXN2 and ATXN3 aggregation, nor with their protein levels.

G3BP1 Overexpression Reduces the Number of Cells with Aggregates and the Levels of ATXN2 and ATXN3 Proteins

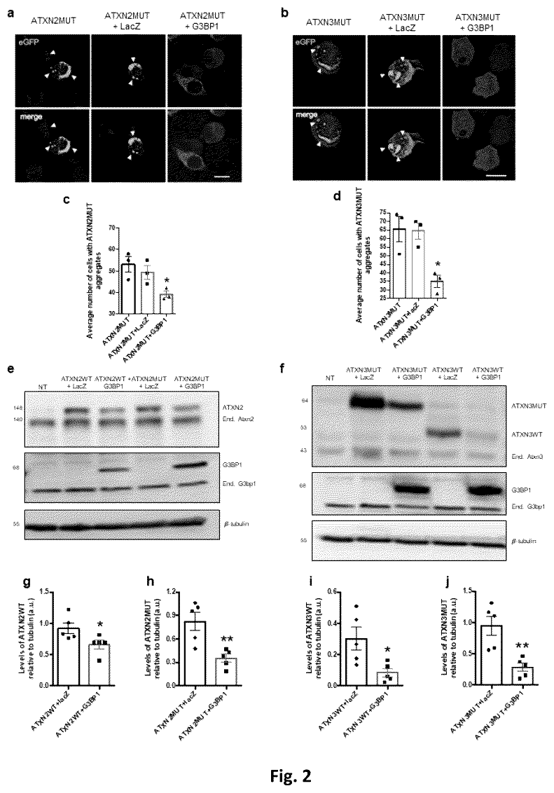

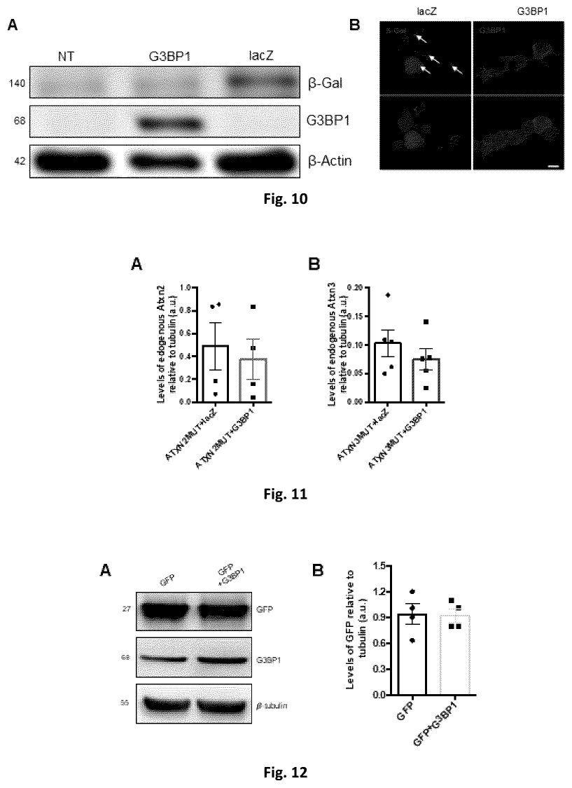

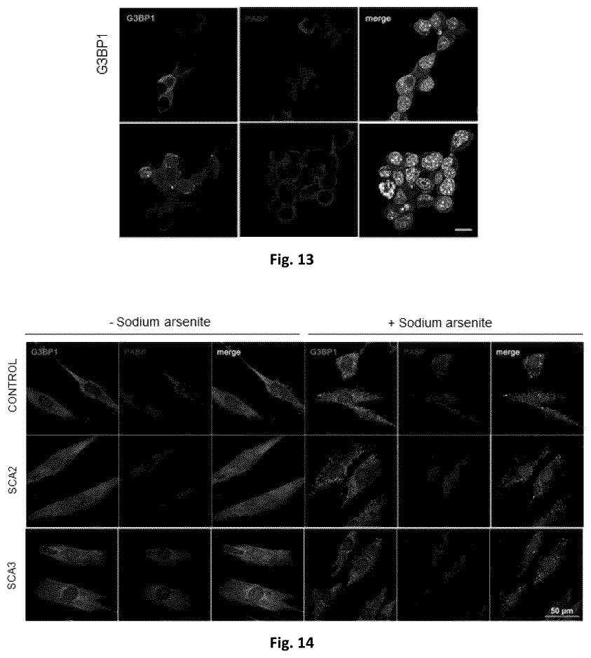

SGs assembly can also be induced by overexpression of its core components^{13,31}, including G3BP1, which is an RBP able of both mRNA stabilization and degradation¹⁵. However, in the present disclosure it was observed that in Neuro2a G3BP1 overexpression alone is less effective in inducing SGs formation, than when combining it with a sodium arsenite stimulus (FIG. 10). In this line, in fibroblasts from SCA2 and SCA3 patients, G3BP1 has a diffuse expression, which is also observed in healthy fibroblasts (FIG. 11). On the contrary, upon sodium arsenite treatment G3BP1 condensates, in PABP positive foci (FIG. 11). As observed for SGs, G3BP1 overexpression also leads to an inhibition of protein synthesis, although at lower levels (FIG. 9b, 1d). Taking this into consideration, it was then investigated the impact of G3BP1 overexpression in ATXN2MUT and ATXN3MUT proteins. To achieve this goal, it was co-transfected Neuro2a cells with ATXN2MUT or ATXN3MUT and G3BP1, and as controls cells co-transfected with ATXN2MUT or ATXN3MUT and lacZ, and cells transfected with ATXN2MUT or ATXN3MUT (FIG. 12). As mentioned, in Neuro2a the expression of the mutant forms of both proteins leads to the formation of aggregates, which are a hallmark of polyQ diseases (FIG. 2a, 2b). It was found that G3BP1 overexpression was able to significantly reduce the number of cells with aggregates of both ATXN2MUT (ATXN2MUT+G3BP1: 0.39±0.0153, versus ATXN2MUT: 0.53±0.036, n=3, P=0.0233) and ATXN3MUT (ATXN3MUT+G3BP1: 0.35±0.036, versus ATXN3MUT: 0.66±0.073, n=3, P=0.0201), compared to control conditions (FIG. 2c, 2d). Next, it was investigated whether the observed reduction in aggregation upon G3BP1 overexpression could be associated with a reduction in the protein levels of ATXN2MUT and ATXN3MUT (FIG. 2e, 2f). Additionally, it was also analyzed the impact of G3BP1 overexpression in the protein levels of non-pathological forms, respectively ATXN2WT and ATXN3WT (FIG. 2e, 2f). It was found that G3BP1 overexpression was able to significantly reduce the expression levels of both ATXN2WT (ATXN2WT+G3BP1: 0.65±0.06, versus, ATXN2WT+lacZ: 0.692±0.08, n=5, P=0.04) and ATXN2MUT (ATXN2MUT+G3BP1: 0.35±0.1343, versus, ATXN2MUT+lacZ: 0.82±0.116 n=5, P=0.0076) (FIG. 2g, 2h). In the same way, it was also observed a significant reduction in ATXN3WT and ATXN3MUT levels upon G3BP1 overexpression, compared to control conditions (ATXN3WT+G3BP1: 0.608±0.026, versus, ATXN3WT+lacZ: 0.3±0.071, n=5, P=0.02 and ATXN3MUT+G3BP1: 0.28±0.067, versus, ATXN3MUT+lacZ: 0.95±0.154 n=5, P=0.004, respectively) (FIG. 2i, 2j). However, no alteration was observed in mouse endogenous levels of Ataxin-2 and Ataxin-3 upon G3BP1 overexpression (FIG. 13). Moreover, in an additional control experiment, GFP levels were not altered when G3BP1 is overexpressed (FIG. 14). Altogether, these results show that G3BP1 reduces the levels and aggregation of mutant ataxin-2 and mutant ataxin-3 proteins.

### The NTF2-Like Domain is Important in G3BP1 Action on Ataxin-2 and Ataxin-3 Mutant Proteins

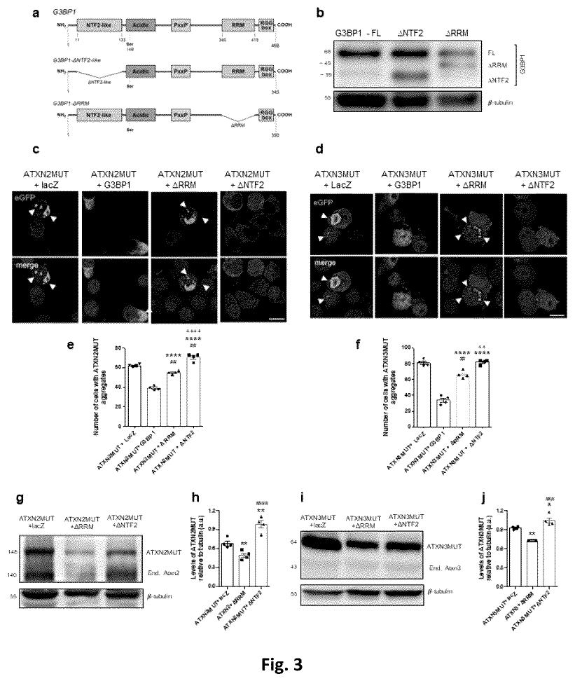

G3BP1 is an RBP with several molecular and biological functions, including mRNA binding, DNA binding³², helicase, and has important functions in immune response³⁴. Overall, RBPs, including G3BP1, interact with mRNAs through specific RNA-binding domains^{35,36}. The RNA recognition motif (RRM) of G3BP1 is known for interacting with target RNA sequences³⁷. G3BP1 also harbors a NTF2-like domain that is involved in the nuclear shuttling of proteins through the nuclear pore complex³⁸, facilitates protein-protein interactions³⁹, mediates G3BP1 dimerization, and is important in SGs formation¹³. Therefore, to better understand G3BP1 action on mutant ataxin-2 and ataxin-3 aggregation and levels, the inventors of the present disclosure developed two different forms of the protein, one with the deletion of the NTF2-like domain (G3BP1-ΔNTF2) and the other with the deletion of the RRM domain (G3BP1-ΔRRM) (FIG. 3a, 3b). Next, it was co-transfected Neuro2a cells with the ATXN2MUT or ATXN3MUT and either G3BP1-ΔNTF2 or G3BP1-ΔRRM, and additionally with full length G3BP1 and lacZ, as controls (FIG. 3c, 3d). The expression of G3BP1-ΔRRM leads to a significant decrease in the number of cells with aggregates of ATXN2MUT and ATXN3MUT, compared to the lacZ control condition (ATXN2MUT+G3BP1-ΔRRM: 55±0.815 versus ATXN2MUT+lacZ: 62±0.814, n=4, P<0.001, and ATXN3MUT+G3BP1-ΔRRM: 66.5±2.305 versus ATXN3MUT+lacZ: 80.7±2.37, n=4, P<0.001, respectively) (FIG. 3e, 3f). However, when compared to the expression of the full length G3BP1, G3BP1-ΔRRM leads to a significant increase in the number of cells with aggregates of ATXN2MUT and ATXN3MUT. On the contrary, the expression of G3BP1-ΔNTF2 leads to an increase of cells with aggregates of ATXN2MUT and ATXN3MUT, compared to both lacZ and full length G3BP1 conditions (FIG. 3e, 3f). Next, it was analyzed the levels of ATXN2MUT and ATXN3MUT upon expression of both truncated forms of G3BP1 (FIG. 3g, 3i). It was found that G3BP1-ΔRRM leads to a significant reduction of the levels of ATXN2MUT and ATXN3MUT, compared to control (ATXN2MUT+G3BP1-ΔRRM: 0.48±0.035 versus, ATXN2MUT+lacZ: 0.64±0.013, n=4, P<0.001, and ATXN3MUT+G3BP1-ΔRRM: 0.725±0.001 versus, ATXN3MUT+lacZ: 0.93±0.012, n=4, P<0.001, respectively) (FIG. 3h, 3j). On the contrary, the expression of G3BP1-ΔNTF2 leads to a significant increase in the levels of ATXN2MUT and ATXN3MUT proteins (FIG. 3h, 3j). Altogether, these results point to a relevant role of NTF2-like domain in important for G3BP1 molecular mechanism of action on mutant ataxin-2 and mutant ataxin-3 proteins.

### Ser149 Phosphorylation Site is Important in G3BP1 Action on Ataxin-2 and Ataxin-3 Mutant Proteins

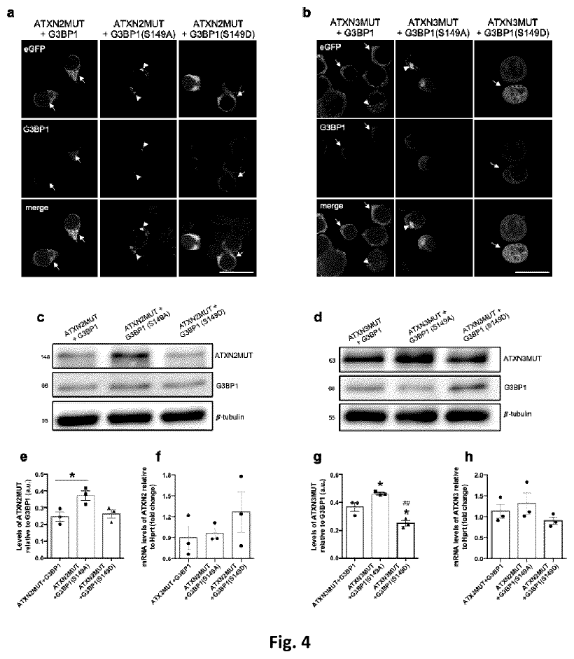

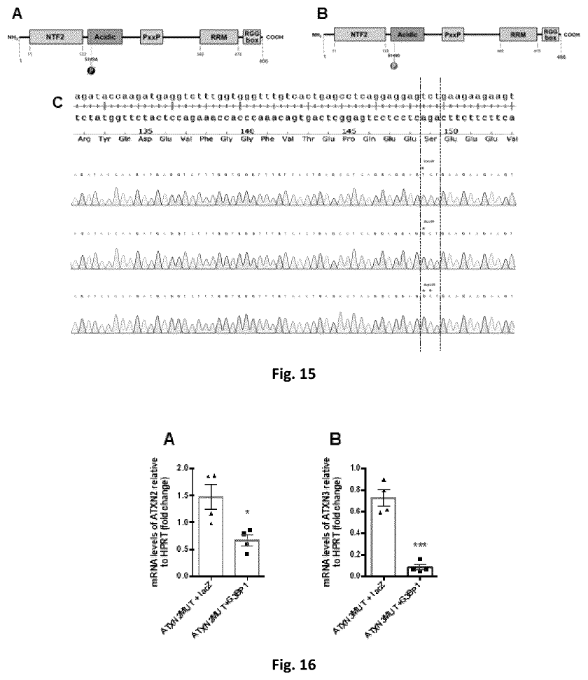

In G3BP1 protein, the NTF2-like domain is closely located to a phosphorylation site (Ser-149), which seems to have an important functional role^{17,36}. The G3BP1-ΔRRM was able to reduce the levels and aggregation of ATXN2MUT and ATXN3MUT, however to a lesser extent than the full length G3BP1. Therefore, the inventors of the present disclosure aimed to investigated the importance of Ser149 in the functional role of G3BP1. For that, it was developed two phosphomutants of G3BP1, a phosphomimetic S149D and a nonphosphorylatable S149A (FIG. 15). Neuro2a cells were co-transfected with ATXN2MUT or ATXN3MUT and G3BP1(S149D) and G3BP1(S140A). With confocal imaging it was observed that in cells expressing wild-type G3BP1 there are no aggregates of ATXN2MUT or ATXN3MUT (FIG. 4a, 4b; white arrows). The same pattern is observed upon expression on the phosphomimetic G3BP1 (S149D). On the contrary, aggregates of ATXN2MUT and ATXN3MUT were observed in cells expressing the phospho-dead G3BP1(S149A) (FIG. 4a, 4b; white arrow heads). Next, it was investigated the impact of the two phosphomutants on the protein levels of ATXN2MUT and ATXN3MUT (FIG. 4c, 4d). It was found that the levels of ATXN2MUT protein are significantly increased with G3BP1(S149A) expression (ATXN2MUT+G3BP1: 0.24±0.026 versus ATXN2MUT+G3BP1(S149A): 0.37±0.028, n=3, P<0.05) (FIG. 4e). On the other hand, the levels of ATXN2MUT protein are similar between wild-type G3BP1 and the phosphomimetic G3BP1(S149D) (FIG. 4e). In the same line, the levels of ATXN3MUT protein are increased upon nonphosphorylatable G3BP1(S149A) expression, compared to wild-type G3BP1 and G3BP1(S149D) conditions (FIG. 4g). The expression of G3BP1(S149D) also leads to a significant reduction in the levels of ATXN3MUT, as compared to wild-type G3BP1 condition (ATXN3MUT+G3BP1: 0.36±0.03 versus ATXN3MUT+G3BP1(S149D): 0.25±0.01, n=3, P<0.05). Upon wild-type G3BP1 expression it was observed that there was a significant reduction in the mRNA levels of ATXN2MUT and ATXN3MUT, compared to control conditions (FIG. 16). However, no differences were observed in the mRNA levels of ATXN2MUT and ATXN3MUT upon the expression of the two phosphomutants, compared to the wild-type G3BP1 (FIG. 4f, 4h). Altogether, these results suggest that Ser-149 phosphorylation site is important for G3BP1 molecular activity, modulating the aggregation and protein levels of mutant ataxin-2 and mutant ataxin-3.

G3BP1 mRNA and protein levels are reduced in SCA2 and SCA3, whereas silencing it increases aggregation in the mouse brain

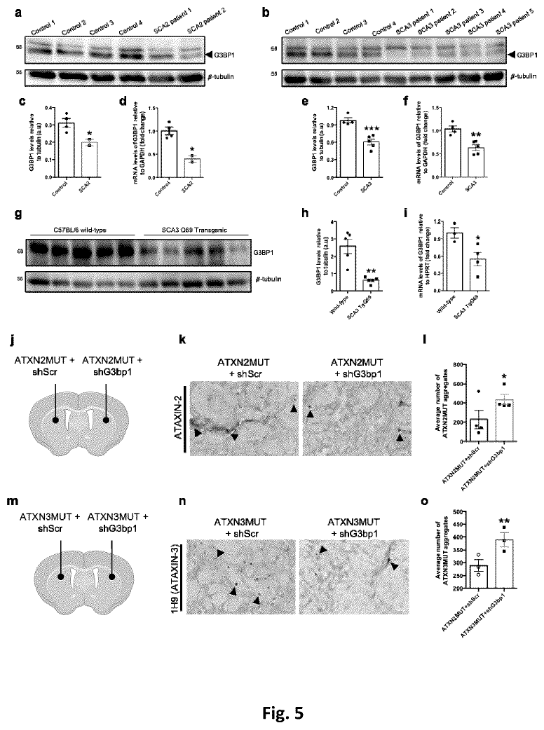

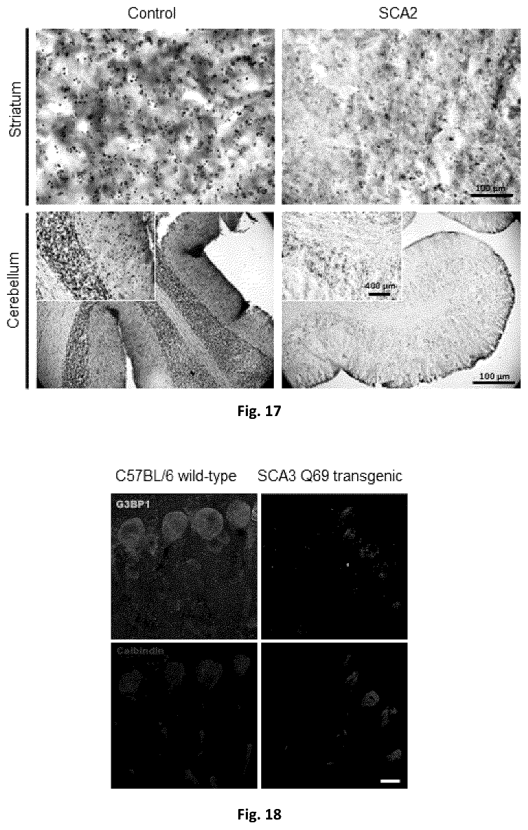

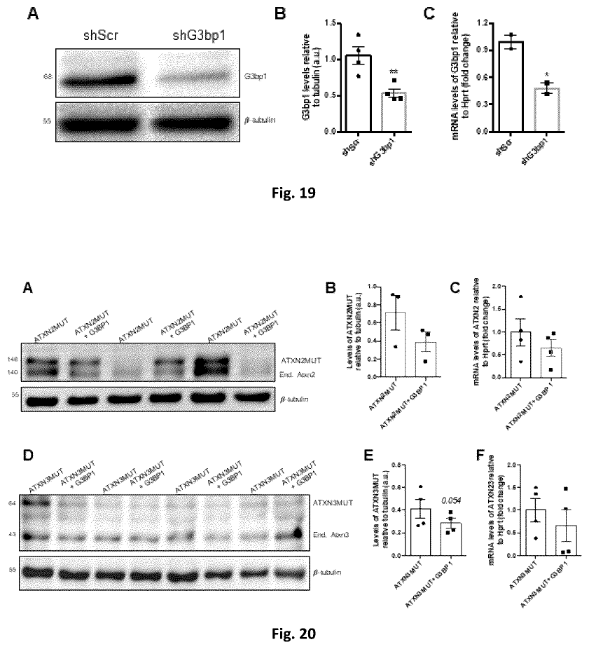

Previous studies report that mutant polyQ proteins can dysregulate the expression of several genes^{1,41}. In fact, the inventors of the present disclosure showed that the expression of mutant ataxin-3 drives an abnormal reduction of wild-type ataxin-2 levels⁴². In this line, it was then analyzed the levels of G3BP1 in samples from SCA2 and SCA3 patients and disease models. In post-mortem brain samples of SCA2 patients it was detected a reduction in the immunodetection of G3BP1, comparing with healthy individuals, both in striatum and cerebellum (FIG. 17). Furthermore, in fibroblasts from SCA2 patients it was detected a significant reduction in the levels of G3BP1 protein (FIG. 5a, 5c) and mRNA (FIG. 5d), compared to fibroblasts from healthy controls. In the same line, in fibroblasts from SCA3 patients it was observed a reduction in G3BP1 protein (FIG. 5b, 5e) and mRNA levels (FIG. 5f), compared to fibroblasts form healthy controls. This reduction was also observed in a transgenic mouse model of SCA3, which was used in this study (FIG. 5g-i). In fact, G3BP1 protein and mRNA levels are significantly reduced in the transgenic SCA3 animals, compared to wild-type C57BL/6. This transgenic mouse expresses a truncated form of ataxin-3 with 69 glutamines in the Purkinje cells of the cerebellum. In fact, the detected reduction of G3BP1 in the transgenic animals is particular evident in these cells (FIG. 18). To investigate the functional impact of G3BP1 reduction, lentiviral vectors encoding a validated shRNA targeting G3bp1 (shG3bp1) (FIG. 19) were injected in the lentiviral rat model of SCA2 and SCA343,44 (FIG. 5i, 5l). Briefly, one hemisphere of the striatum was co-injected with lentiviral vectors encoding for ATXN2MUT (or ATXN3MUT) and the shG3bp1, while in the contralateral hemisphere, as control we injected ATXN2MUT (or ATXN3MUT) and a scramble shRNA (shSrc). At 4 weeks post-injection the animals were sacrificed, and the striatum was histologically analyzed for the presence of aggregates of ATXN2MUT and ATXN3MUT (FIG. 5j, 5m). It was found that, the silencing of G3BP1 leads to a significant increase in the average number of aggregates of ATXN2MUT (ATXN2MUT+shScr: 434±55.62 versus ATXN2MUT+shG3bp1: 228±98.85, n=4, P<0.01) and ATXN3MUT (ATXN3MUT+shScr: 390±26.89 versus ATXN3MUT+shG3bp1: 290±22.37, n=3, P<0.05) (FIG. 5k, 5n). Altogether, these results highlight that in SCA2 and SCA3 the reduced levels of G3BP1 mRNA and protein are important for disease pathogenesis.

### Restoring G3BP1 Levels Alleviates Neuropathology in SCA2 and SCA3 Lentiviral Mouse Models

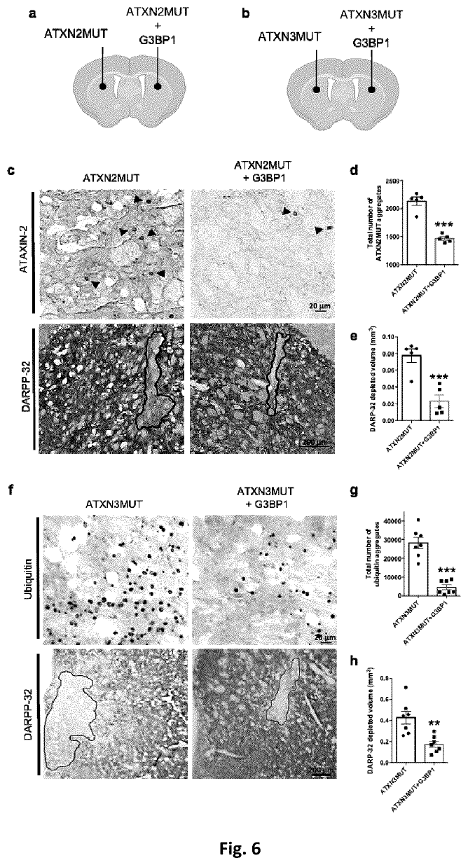

The expression of ATXN2MUT and ATXN3MUT, mediated by lentiviral vectors leads to the formation of intraneuronal aggregates and to the loss of neuronal markers^{43,44}, which are neuropathological signs also found in post-mortem human tissue⁴⁵-47. Thus, the inventors of the present disclosure investigated whether restoring G3BP1 levels improve neuropathological abnormalities induced by ATXN2MUT and ATXN3MUT in vivo. For that lentiviral vectors encoding ATXN2MUT (or ATXN3MUT) and human G3BP1 were co-expressed in one hemisphere of the striatum and, as a control, in the contralateral hemisphere, lentiviral vectors encoding ATXN2MUT (or ATXN3MUT) were injected (FIG. 6a, 6b). At 12 weeks post-injection for the SCA2 lentiviral mouse model, and at 4 weeks post-injection for the SCA3 lentiviral mouse model, the animals were sacrificed, and the striatum was histologically analyzed. In both models, the expression of G3BP1 was able to significantly reduce the number of ATXN2MUT aggregates (ATXN2MUT+G3BP1: 1466±31.13, n=5, versus ATXN2MUT: 2131±71.04, n=5, P=0.0002) and ATXN3MUT aggregates (ATXN3MUT+G3BP1: 6066±1958, versus ATXN3MUT: 30076±2717, n=7, P<0.0001) (FIG. 6c-d, 6f-g). The mRNA and soluble protein levels of ATXN2 and ATXN3 were also analyzed in a group of animals at 4 weeks post-injection (FIG. 20). In the SCA2 lentiviral model, no significant differences were observed in mRNA and protein levels of ATXN2MUT upon G3BP1 expression (FIG. 20a-b). On the other hand, in the SCA3 lentiviral model, there is a robust reduction in ATXN3MUT protein levels in the hemisphere expressing G3BP1 (ATXN3MUT+G3BP1: 0.285±0.04, versus ATXN3MUT: 0.413±0.08, n=4, P=0.054) (FIG. 20d-e). No alterations in the ATXN2MUT or ATXN2MUT mRNA levels were observed between hemispheres (FIG. 20c, 20f). The expression of mutant ataxin-2 or mutant ataxin-3 in the striatum leads to the loss of neuronal markers43,44. Accordingly, to the above results, G3BP1 expression led to a preservation of neuronal marker DARPP-32 in both models, as compared to the control hemispheres (ATXN2MUT+G3BP1: 0.02±0.0078, versus ATXN2MUT: 0.08±0.0078, n=5, P=0.001; ATXN3MUT+G3BP1: 0.19±0.0291, versus ATXN3MUT: 0.45±0.0647, n=7, P=0.0072) (FIG. 6c, 6e, 6f, 6h). Altogether these results show that G3BP1 expression in the striatum mediates neuroprotection, reducing neuropathological hallmarks associated with the expression of mutant ataxin-2 and mutant ataxin-3.

### Overexpression of G3BP1 in the Brain of Wild-Type Mice Did not Produce Neuronal Loss Nor Astrogliosis

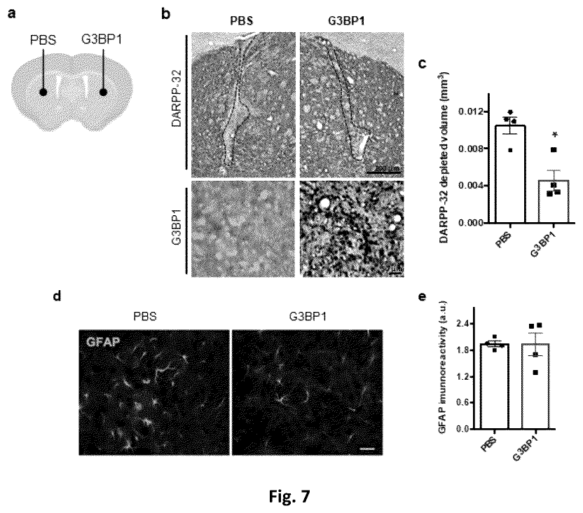

Based on the previous results, the inventors of the present disclosure evaluated the impact of G3BP1 expression in the brain of wild-type animals. For that, lentiviral particles encoding G3BP1 were injected in one hemisphere of the striatum of wild-type C57BL/6 mice, while in the contralateral hemisphere was injected with PBS, as control (FIG. 7a). At 4 weeks post-injection the loss of the neuronal marker DARPP-32 (FIG. 7b) in the hemisphere injected with G3BP1 was significantly smaller comparing to the control hemisphere injected with PBS (G3BP1: 0.003±0.0014, versus PBS: 0.01±0.0011, n=4 Student's t-test, P=0.035) (FIG. 7b, 7c). In fact, in the G3BP1-injected animals the lesion area was restricted to the injection site. In this line, it was also analyzed the astrocytes activation through GFAP marker comparing the G3BP1 injected hemisphere to the control PBS-injected hemisphere (FIG. 7d). No differences were found in the immunoreactivity of GFAP between both hemispheres (FIG. 7e). Altogether these results point that G3BP1 overexpression in the normal brain does not seem to produce toxicity.

### Restoring G3BP1 Levels in the Cerebellum Mitigates Behavior Deficits and Neuropathological Abnormalities in a SCA3 Transgenic Mouse Model.

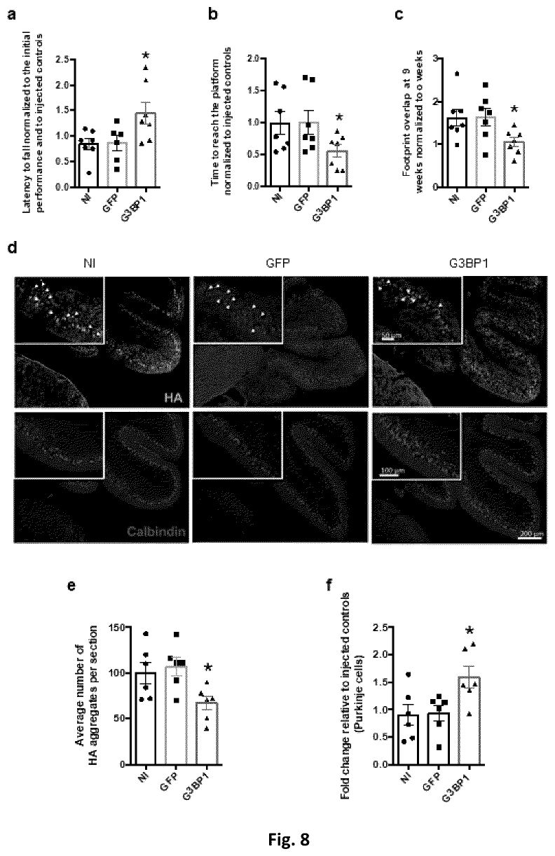

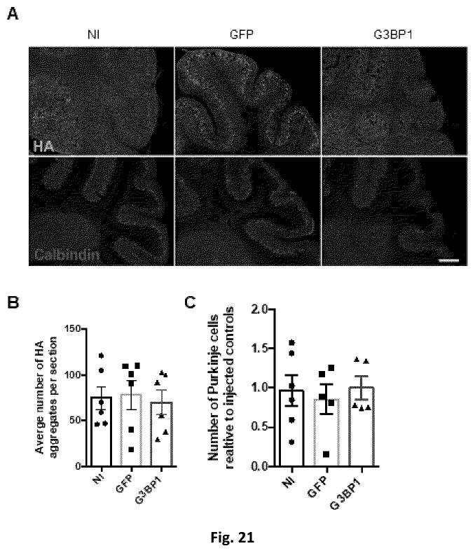

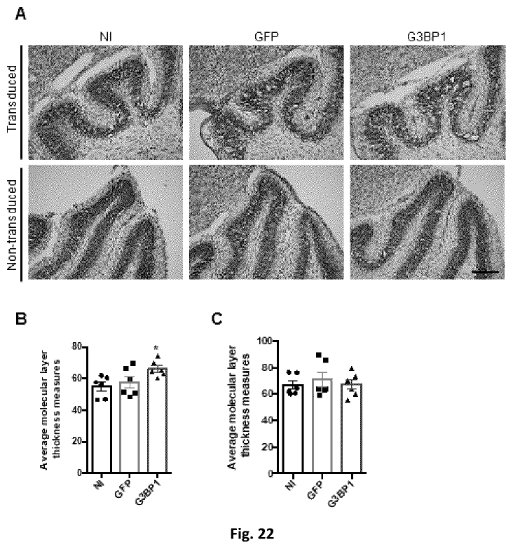

PolyQ SCAs are characterized by a progressive neuronal loss and motor dysfunctionality. Thus, to mimic this phenotype, in the present disclosure a transgenic mouse model expressing a truncated form of mutant ataxin-3 with 69 glutamines was used and characterized by a severe motor dysfunctions, neurodegeneration and early onset²³. This can also be a relevant polyQ model, considering that only contains a small region of the ataxin-3 protein, and a significant tract of glutamines, causing pathology, as observed in other polyQ diseases^{23,48}. Therefore, it was then investigated the impact of G3BP1 expression in this transgenic mouse model, which has reduced levels of G3BP1 (FIG. 5g-i). For that, at 4 weeks of age, animals were stereotaxically injected into cerebellum⁴⁹ with lentiviral vectors encoding for G3BP1, while control animals were injected with lentiviral vectors encoding for GFP. A third group of non-injected animals was also used. After, the animals were subjected to a battery of behavior tests every 3 weeks, until 9 weeks post-injection. At this final timepoint, G3BP1-injected animals stayed for longer times in the rotating rotarod, comparing to the control animals, thus showing an improvement in motor deficits (G3BP1: 1.45±0.0124, n=7, versus NI: 0.84±0.1082, n=7, P=0.0254) (FIG. 8a). In the same line, at 9 weeks post-injection, in the swimming test, in which the animals have to cross a pool to reach a safe platform, G3BP1-injected animals took less time to reach the platform, compared to control animals (G3BP1: 0.55±0.0974, n=7, versus NI: 0.99±0.173, n=7, P=0.0476) (FIG. 8b). Finally, in the footprint patterns test, in which the animals cross a white sheet tunnel with their paws painted, the animals injected with G3BP1 had a smaller footprint overlap, as compared to control animals, which suggests an improvement in motor deficits (G3BP1: 1.06±0.1081, n=7, versus NI: 1.62±0.1997, n=7, P=0.0297) (FIG. 8c). Altogether, these results show that G3BP1 expression in the cerebellum can ameliorate motor deficits. Neuropathologically, this mouse model is characterized by the formation of aggregates in the Purkinje cells of the cerebellum, showing a high reduction in the number of these cells and a strong disarrangement of cerebellar layers architecture^{23,50}. Thus, it was then evaluated the impact of G3BP1 expression in the neuropathological abnormalities (FIG. 8d). Accordingly with the improvements observed in motor deficits, it was found that animals injected with G3BP1 exhibited significantly reduced the number of pathological aggregates (HA tagged), compared to controls (G3BP1: 63.12±10.17, n=6, versus NI: 101.2±15.29, n=6, P=0.0397) (FIG. 8e). As the expression of ATXN3MUT in the model is directed to the Purkinje cells of the cerebellum, it was also evaluated their number using calbindin marker. It was found that G3BP1-injected animals showed a preservation in the number of Purkinje cells, compared to controls (G3BP1: 1.62±0.2405, n=6, versus NI: 0.91±0.1904, n=6, P=0.0437) (FIG. 8f). Importantly, in non-transduced lobes no differences were found between the experimental groups regarding pathological aggregates (G3BP1: 44.45±7.169, n=6, versus NI: 49.7±9.385, n=6) or the number of cerebellar Purkinje cells (G3BP1: 1±0.1457, n=6, versus NI: 0.97±0.1988 n=6) (FIG. 21). As these transgenic animals present a strong atrophy of the cerebellum, it was then analyzed the thickness of cerebellum layers. It was found that the molecular layer thickness of transduced lobules (II/III) was significantly wider compared to non-injected controls (G3BP1: 64.99±3.189, n=6 versus NI: 56.03±1.824, n=6, P<0.0118), while no difference was found in non-transduced lobules (FIG. 22). Altogether, these results show that G3BP1 expression in the cerebellum significantly reduced motor behaviour impairments and reduces the neuropathological abnormalities.

### Discussion

Proteins containing abnormally expanded polyQ tracts have been implicated with the impairment of several cellular pathways, which ultimately lead to cellular dead. The high propensity of the mutant polyQ proteins to aberrantly aggregate are either directly involved or at least contribute to aggravate particular toxic outcomes, acting decisively in the polyQ pathogenesis. In the last decade it has been hypothesized that the abnormal protein aggregation, characteristic of several neurodegenerative disorders, not only subjects cells to stress, but can also impair cellular stress-response pathways⁵¹. The formation of stress granules is one important player in stress response, as they play an important role as mediator of protein synthesis. During SGs assembly, several key players, such as RBPs and mRNAs are sequestered into the granule preventing these components from integrating the translational machineryl^{2,52}. Previous evidence has also shown that SGs co-localize with several protein aggregates, which are characteristic of different neurodegenerative diseases⁵³. Therefore, the inventors of the present disclosure hypothesized that an activation of the stress response through the formation of SGs could either sequestrate mutant polyQ proteins, or promote a translational arrest, decreasing its expression. It was found that chemically inducing SGs assembly, in Neuro2a cells expressing either mutant ataxin-2 our mutant ataxin-3, leads to a significant decrease in global translation levels, however it seems not to interfere with the expression levels of both ATXN2MUT and ATXN3MUT, nor with the aggregation of those proteins.

G3BP1 is an RBP, a core component of SGs and in its dephosphorylated state can induce SGs formation¹³. It has been reported that cellular stress induction by sodium arsenite, reduces the constitutive phosphorylation state of G3BP113,54. However, in recent years, this hypothesis was challenged⁵⁴, and it is not clear if there is a correlation between cellular stress induction through sodium arsenite and phosphorylation/dephosphorylation status of G3BP1. To clarify this possible link, the inventors of the present disclosure overexpressed G3BP1 in SCA2 patients-derived fibroblasts. It was found that G3BP1 shows a diffuse expression within the cell, contrasting to what happens when we treat the cells with sodium arsenite. Upon sodium arsenite treatment, G3BP1 self-assembles in structures resembling SGs. As G3BP1 functions vary depending on its phosphorylation/dephosphorylation state, the next aim was to study the impact of G3BP1 overexpression in Neuro2a cells expressing ATXN2MUT e ATXN3MUT. Upon overexpression of G3BP1 it was observed a reduction in the number of cells with mutant protein aggregates and in the expression levels of mutant polyQ proteins. It was hypothesized that, while the phosphorylated G3BP1, diffusely spread in the cells, performs its catalytic activity on the mutant polyQ proteins, the non-phosphorylated G3BP1 assembles in SGs-like structures, switching its functions.

To clarify the specificity of G3BP1 action on ATXN2MUT and ATXN3MUT levels and aggregates, in the present disclosure Neuro2a cells with low levels of mouse endogenous G3bp1 were used. It was observed that when ATXN2MUT and ATXN3MUT are expressed in this cell line, the number of cells with aggregates is maintained, comparing to a normal Neuro2a cell line. However, when co-express G3BP1 and the mutant proteins are co expressed, the results are in line to what is observed in the in Neuro2a cells, i.e., a decrease in the number of cells with aggregates. These observations lead to suggest that is G3BP1 expression the responsible for the decrease of the levels and number of aggregates of mutant polyQ proteins.

Next, the inventors investigated which domains of G3BP1 could be implicated in its molecular mechanisms of action. Thus, the study was focused in the NTF2-like domain, which is involved in the nuclear transport via nuclear pores and has been shown to facilitate protein-protein interactions⁵⁵. Additionally, it was also investigated the contribution of the RRM domain, which interacts with target RNA sequences and can also bind with other proteins⁵⁶. By expressing truncated constructs of G3BP1 with deletions of NTF2 or RRM domains, it was observed a reduction in the number of cells with aggregates of both ATXN2MUT and ATXN3MUT and their expression levels, when the RRM domain is deleted. Opposingly, no differences were found among the experimental conditions when NTF2 is deleted. This led to suggest that NTF2 domain could be essential for G3BP1 action. The inventors next went to analyze the impact of G3BP1 expression on the mRNA levels of ATXN2MUT and ATXN3MUT. It was found that those levels were significantly decreased upon G3BP1 expression. G3BP1 protein was found to interact with ATXN3 RNA⁴⁰, which could be the cause for the more robust results found in ATXN3 mRNA, comparing with ATXN2. Previous studies demonstrated that phosphorylated G3BP1 translocates to the cellular nucleus, probably to perform its endoribonuclease activityl^{7,33}. As mentioned before, the NTF2-like domain of G3BP1 is very close to an important phosphorylation site, serine 149. This phosphorylation site is also believed to be connected to the endonuclease activity of G3BP133. To evaluate the impact of G3BP1 phosphorylation it was performed a sited-directed mutagenesis in G3BP1, switching the serine149 for an alanine, therefore generating a phospho-dead protein at the 149 aa site. Using this phospho-dead construct it was found that the expression of G3BP1 lost its impact in the number of cells with aggregates of ATXN2MUT and ATXN3MUT, leading us to suggest that G3BP1 phosphorylation is crucial for its molecular functions.

Next, the inventors of the present disclosure analyzed G3BP1 expression levels in the context of SCA2 and SCA3 patients and animal models. It was found that in postmortem samples of human brain tissue from SCA2, G3BP1 staining was substantially decreased, suggesting low levels of expression. Accordingly, it was also observed that SCA2 and SCA3 patient-derived fibroblasts have reduced levels of G3BP1 mRNA and protein. The results showed that molecular pathologic phenotype observed in SCA2 and SCA3 is exacerbated due to the joint effect of the polyQ mutant proteins toxicity and the lower expression levels of G3BP1. Therefore, it was then investigated the potential of G3BP1 re-establishment in disease mitigation, using different mice models of these two diseases. Using SCA2 and SCA3 lentiviral mouse models, it was observed that the injection into the striatum of lentiviral particles encoding for G3BP1 lead to the preservation of brain tissue (DARPP-32 staining) and to a decrease in the number of aggregates. Moreover, in a transgenic mouse model, characterized by severe neurodegeneration and motor deficits, it was found that the injection in the cerebellum of lentiviral particles encoding for G3BP1, reduces the number of aggregates and preserves the number of Purkinje cells. Importantly, G3BP1 expression in mice cerebella significantly improved the overall motor performance, balance, and coordination.

With the present disclosure it was surprisingly found that G3BP1 expression levels are decreased in both patient-derived fibroblast and brain sample of SCA2 and SCA3 affected individuals. Additionally, it was shown that G3BP1 expression can decrease the expression of mutant ataxin-2 and ataxin-3. These results strongly support that, in SCA2 and SCA3 disease, the ability of G3BP1 to downregulate the mutant ataxin-2 and ataxin-3 is impaired, due to G3BP1 decreased expression levels, leading to an exacerbation of the phenotype. Additionally, it was also shown that the G3BP1 NTF2-like domain and the ser 149 phosphorylation site, are essential to mitigate mutant ataxin-2 and mutant ataxin-3 aggregation.

The results of the present disclosure strongly support that gene delivery of G3BP1 is efficient and safe in the mitigation SCA2 and SCA3 pathology, supporting G3BP1 as a novel therapeutic target, not only for SCA2 and SCA3, but to other polyQ diseases.

The term “comprising” whenever used in this document is intended to indicate the presence of stated features, integers, steps, components, but not to preclude the presence or addition of one or more other features, integers, steps, components or groups thereof.

The disclosure should not be seen in any way restricted to the embodiments described and a person with ordinary skill in the art will foresee many possibilities to modifications thereof. The above described embodiments are combinable.

The following dependent claims further set out particular embodiments of the disclosure.

### REFERENCES

1. Matos, C. A., de Almeida, L. P. & Nóbrega, C. Machado-Joseph disease/spinocerebellar ataxia type 3: lessons from disease pathogenesis and clues into therapy. Journal of Neurochemistry 148, 8-28 (2019).
2. Pulst, S.-M., Nechiporuk, A. & Nechiporuk, T. Moderate expansion of a normally biallelic trinucleotide repeat in spinocerebellar ataxia type 2. Nat. Genet. 14, 269-276 (1996).
3. Kawaguchi, Y. et al. CAG expansions in a novel gene for Machado-Joseph disease at chromosome 14q32.1. Nat. Genet. 8, 221-228 (1994).
4. Matos, C., Macedo-Ribeiro, S. & Carvalho, A. Polyglutamine diseases: The special case of ataxin-3 and Machado-Joseph disease. Prog. Neurobiol. 95, 26-48 (2011).
5. Matos, C., Pereira de Almeida, L. & Nobrega, C. Proteolytic Cleavage of Polyglutamine Disease-Causing Proteins: Revisiting the Toxic Fragment Hypothesis. Curr. Pharm. Des. 23, 753-775 (2017).
6. Huynh, D. P., Figueroa, K., Hoang, N. & Pulst, S. M. Nuclear localization or inclusion body formation of ataxin-2 are not necessary for SCA2 pathogenesis in mouse or human. Nat. Genet. 26, 44-50 (2000).
7. Onodera, O. et al. Progressive atrophy of cerebellum and brainstem as a function of age and the size of the expanded CAG repeats in the MJD1 gene in Machado-Joseph disease. Ann. Neurol. 43, 288-296 (1998).
8. Todd, T. W. & Lim, J. Aggregation formation in the polyglutamine diseases: protection at a cost? Mol. Cells 36, 185-194 (2013).
9. Cowan, K. J., Diamond, M. I. & Welch, W. J. Polyglutamine protein aggregation and toxicity are linked to the cellular stress response. Hum. Mol. Genet. 12, 1377-1391 (2003).
10. Takahashi, A. & Ohnishi, T. Molecular mechanisms involved in adaptive responses to radiation, UV light, and Heat. Journal of Radiation Research 50, 385-393 (2009).
11. Kedersha, N. & Anderson, P. Stress granules: sites of mRNA triage that regulate mRNA stability and translatability. Biochem. Soc. Trans. 30, 963-969 (2002).
12. Marcelo, A., Koppenol, R., de Almeida, L., Matos, C. & Nóbrega, C. Stress granules, RNA-binding proteins and Polyglutamine diseases: too much aggregation? Cell Death Dis. (2021). doi: 10.1038/s41419-021-03873-8
13. Tourrière, H. et al. The RasGAP-associated endoribonuclease G3BP assembles stress granules. J. Cell Biol. 160, 823-831 (2003).
14. Sahoo, P. K. et al. Axonal G3BP1 stress granule protein limits axonal mRNA translation and nerve regeneration. Nat. Commun. 9, 3358 (2018).
15. Winslow, S., Leandersson, K. & Larsson, C. Regulation of PMP22 mRNA by G3BP1 affects cell proliferation in breast cancer cells. Mol. Cancer 12, 156 (2013).
16. Martin, S. et al. Preferential binding of a stable G3BP ribonucleoprotein complex to intron-retaining transcripts in mouse brain and modulation of their expression in the cerebellum. J. Neurochem. 139, 349-368 (2016).
17. Gallouzi, I. et al. A Novel Phosphorylation-Dependent RNase Activity of GAP-SH3 Binding Protein: a Potential Link between Signal Transduction and RNA Stability. Mol. Cell. Biol. 18, 3956-3965 (1998).
18. Chai, Y., Shao, J., Miller, V. M., Williams, A. & Paulson, H. L. Live-cell imaging reveals divergent intracellular dynamics of polyglutamine disease proteins and supports a sequestration model of pathogenesis. Proc. Natl. Acad. Sci. U.S.A. 99, 9310-9315 (2002).
19. Huynh, D. P. Expansion of the polyQ repeat in ataxin-2 alters its Golgi localization, disrupts the Golgi complex and causes cell death. Hum. Mol. Genet. 12, 1485-1496 (2003).
20. Nóbrega, C. et al. The cholesterol 24-hydroxylase activates autophagy and decreases mutant huntingtin build-up in a neuroblastoma culture model of Huntington's disease. BMC Res. Notes 13, 210 (2020).
21. Nóbrega, C. et al. Overexpression of mutant ataxin-3 in mouse cerebellum induces ataxia and cerebellar neuropathology. Cerebellum 12, 441-55 (2013).
22. Xia, G. et al. Generation of human-induced pluripotent stem cells to model spinocerebellar ataxia type 2 in vitro. J. Mol. Neurosci. 51, 237-248 (2013).
23. Torashima, T. et al. Lentivector-mediated rescue from cerebellar ataxia in a mouse model of spinocerebellar ataxia. EMBO Rep. 9, 393-399 (2008).
24. Deglon, N. et al. Self-inactivating lentiviral vectors with enhanced transgene expression as potential gene transfer system in Parkinson's disease. Hum. Gene Ther. 11, 179-190 (2000).
25. Almeida, L. P. De, Ross, C. A., Zala, D., Aebischer, P. & Déglon, N. Lentiviral-Mediated Delivery of Mutant Huntingtin in the Striatum of Rats Induces a Selective Neuropathology Modulated by Polyglutamine Repeat Size, Huntingtin Expression Levels, and Protein Length. J. Neurosci. 22, 3473-3483 (2002).
26. Simões, A. T. et al. Calpastatin-mediated inhibition of calpains in the mouse brain prevents mutant ataxin 3 proteolysis, nuclear localization and aggregation, relieving Machado-Joseph disease. Brain 135, 2428-2439 (2012).
27. Anderson, P. & Kedersha, N. Stressful initiations. J. Cell Sci. 115, 3227-3234 (2002).
28. Mahboubi, H. & Stochaj, U. Cytoplasmic stress granules: Dynamic modulators of cell signaling and disease. BBA—Mol. Basis Dis. 1863, 884-895 (2017).
29. Nonhoff, U., Ralser, M., Welzel, F. & Piccini, I. Ataxin-2 interacts with the DEAD/H-Box RNA helicase DDX& and interferes with P-bobies ans stress granules. Mol. Biol. Cell 18, 1385-1396 (2007).
30. Kedersha, N., Gupta, M., Li, W., Miller, I. & Anderson, P. RNA-binding proteins TIA-1 and TIAR link the phosphorilation of elF-2 alpha to the assembly of mammalian stress granules. J. Cell Biol. 147, 1431-1441 (1999).
31. Protter, D. S. W. & Parker, R. Principles and Properties of Stress Granules. Trends Cell Biol. xx, 1-12 (2016).
32. Liu, Z. S. et al. G3BP1 promotes DNA binding and activation of cGAS. Nat. Immunol. 20, 18-28 (2019).
33. Tourrière, H. et al. RasGAP-Associated Endoribonuclease G3BP: Selective RNA Degradation and Phosphorylation-Dependent Localization. Mol. Cell. Biol. 21, 7747-7760 (2001).
34. Kim, S. S. Y., Sze, L. & Lam, K. P. The stress granule protein G3BP1 binds viral dsRNA and RIG-I to enhance interferon-β response. J. Biol. Chem. 294, 6430-6438 (2019).
35. Lunde, B. M., Moore, C. & Varani, G. RNA-binding proteins: Modular design for efficient function. Nature Reviews Molecular Cell Biology 8, 479-490 (2007).
36. Alam, U. & Kennedy, D. Rasputin a decade on and more promiscuous than ever? A review of G3BPs. Biochim. Biophys. Acta-Mol. Cell Res. 1866, 360-370 (2019).
37. Nagai, K., Oubridge, C., Ito, N., Avis, J. & Evans, P. The RNP domain: a sequence-specific RNA-binding domain involved in processing and transport of RNA. Trends in Biochemical Sciences 20, 235-240 (1995).
38. Vognsen, T., Møller, I. R. & Kristensen, O. Crystal structures of the human G3BP1 NTF2-like domain visualize FxFG Nup repeat specificity. PLOS One 8, (2013).
39. Kennedy, D. et al. Characterization of G3BPs: Tissue specific expression, chromosomal localisation and rasGAP120 binding studies. J. Cell. Biochem. 84, 173-187 (2002).
40. Lin, Y. et al. RNAInter in 2020: RNA interactome repository with increased coverage and annotation. Nucleic Acids Res. 48, D189-D197 (2020).
41. Pflieger, L. T. et al. Gene co-expression network analysis for identifying modules and functionally enriched pathways in SCA2. Hum. Mol. Genet. 26, 3069-3080 (2017).
42. Nóbrega, C. et al. Re-establishing ataxin-2 downregulates translation of mutant ataxin-3 and alleviates Machado-Joseph disease. Brain 138, 3537-3554 (2015).
43. Alves, S. et al. Striatal and nigral pathology in a lentiviral rat model of Machado-Joseph disease. Hum. Mol. Genet. 17, 2071-2083 (2008).
44. Marcelo, A. et al. Autophagy in Spinocerebellar ataxia type 2, a dysregulated pathway, and a target for therapy. Cell Death Dis. (2021).
45. Estrada, R., Galarraga, J., Orozco, G., Nodarse, A. & Auburger, G. Spinocerebellar ataxia 2 (SCA2): Morphometric analyses in 11 autopsies. Acta Neuropathol. 97, 306-310 (1999).
46. Paulson, H. L. et al. Intranuclear Inclusions of Expanded Polyglutamine Protein in Spinocerebellar Ataxia Type 3. Neuron 19, 333-344 (1997).
47. Figiel, M., Szlachcic, W. J., Switonski, P. M., Gabka, A. & Krzyzosiak, W. J. Mouse models of polyglutamine diseases: Review and data table. Part I. Molecular Neurobiology 46, 393-429 (2012).
48. Matos, C., Pereira de Almeida, L. & Nóbrega, C. Machado-Joseph disease/Spinocerebellar ataxia type 3:lessons from disease pathogenesis and clues into therapy. J. Neurochem. (2018). doi:10.1111/jnc.14541
49. Nóbrega, C. et al. Silencing Mutant Ataxin-3 Rescues Motor Deficits and Neuropathology in Machado-Joseph Disease Transgenic Mice. PLOS One 8, e52396 (2013).
50. Oue, M. et al. Characterization of mutant mice that express polyglutamine in cerebellar Purkinje cells. Brain Res. 1255, 9-17 (2009).
51. Duennwald, M. L. Cellular stress responses in protein misfolding diseases. Futur. Sci. OA 1, (2015).
52. Anderson, P. & Kedersha, N. Stress granules: the Tao of RNA triage. Trends Biochem. Sci. 33, 141-150 (2008).
53. Bentmann, E., Haass, C. & Dormann, D. Stress granules in neurodegeneration—lessons learnt from TAR DNA binding protein of 43 kDa and fused in sarcoma. FEBS J. 280, 4348-4370 (2013).
54. Panas, M. D. et al. Phosphorylation of G3BP1-S149 does not influence stress granule assembly. J. Cell Biol. 218, 2425-2432 (2019).
55. Kennedy, D. et al. Characterization of G3BPs: tissue specific expression, chromosomal localisation and rasGAP(120) binding studies. J. Cell. Biochem. 84, 173-187 (2001).
56. Cléry, A., Blatter, M. & Allain, F. H.-T. RNA recognition motifs: boring? Not quite. Curr. Opin. Struct. Biol. 18, 290-298 (2008).

1-20. (canceled)

21. A method of treating a nervous system disease in a subject, the method comprising administering to the subject an isolated or artificial nucleotide encoding the protein G3BP1, wherein the nucleotide comprises a sequence at least 95% identical to the sequence selected from the group consisting of: SEQ ID NO:1; SEQ ID NO:2; SEQ ID NO:3, SEQ ID NO:4; SEQ ID NO:5; SEQ ID NO:6; SEQ ID NO:7 and mixtures thereof.

22. The method of claim 21, wherein the nucleotide comprises a sequence identical to the sequence selected from the group consisting of: SEQ ID NO:1; SEQ ID NO:2; SEQ ID NO:3, SEQ ID NO:4; SEQ ID NO:5; SEQ ID NO:6; SEQ ID NO:7, and mixtures thereof.

23. The method of claim 21, wherein the nervous system disease is a central nervous system disease or a peripherical nervous system disease.

24. The method of claim 21, wherein the nervous system disease is a neurodegenerative disease.

25. The method of claim 21, wherein the nervous system disease is a movement disorder.

26. The method of claim 24, wherein the neurodegenerative disease is a polyglutamine disease.

27. The method of claim 26, wherein said polyglutamine disease is positively influenced by a control of protein aggregation.

28. The method of claim 27, wherein said control of protein aggregation is the control of protein aggregation caused by an expansion in the polyglutamine segment of affected proteins.

29. The method of claim 26, wherein the polyglutamine disease is selected from the group consisting of: Huntington's disease (HD), Spinal bulbar muscular atrophy (SBMA), Dentatorubral-pallidoluysian atrophy (DRPLA), and polyglutamine repeat spinocerebellar ataxia.

30. The method of claim 29, wherein the polyglutamine repeat spinocerebellar ataxia is selected from the group consisting of: spinocerebellar ataxia type 1 (SCA1), Spinocerebellar ataxia type 2 (SCA2), Spinocerebellar ataxia type 3 (SCA3), Spinocerebellar ataxia type 6 (SCA6), Spinocerebellar ataxia type 7 (SCA7) and Spinocerebellar ataxia type 17 (SCA17).

31. The method of claim 21, wherein said nucleotide is administered directly into the brain of the subject or into the spinal cord of the subject.

32. The method of claim 21, wherein said nucleotide is administered by intravascular, intravenous, intranasal, intraventricular or intrathecal injection.

33. The method of claim 21, wherein the nucleotide is a part of a vector or construct.

34. The method of claim 33, wherein the vector or construct is selected from the group consisting of an adenovirus, lentivirus, retrovirus, herpesvirus and Adeno-Associated Virus (AAV) vector.

35. The method of claim 33, wherein the vector or construct is a lentiviral vector.

36. The method of claim 33, wherein the vector or construct is an AAV vector.

37. The method of claim 33, wherein the vector or construct is in a host cell.

38. A method of treating a nervous system disease in a subject, the method comprising administering to the subject a protein encoded by an isolated or artificial nucleotide, wherein the nucleotide comprises a sequence at least 95% identical to the sequence selected from the group consisting of: SEQ ID NO:1; SEQ ID NO:2; SEQ ID NO:3, SEQ ID NO:4; SEQ ID NO:5; SEQ ID NO:6; SEQ ID NO:7 and mixtures thereof.

39. The method of claim 21, wherein the nucleotide is a part of a pharmaceutical composition.


SEQUENCE LISTING (17 sequences)

SEQ ID NO: 1 - DNA, 1401 bp, Artificial Sequence
  Description of Artificial Sequence Synthetic polynucleotide
  G3BP1
atggtgatggagaagcctagtcccctgctggtcgggcgggaatttgtgagacagtattacacactgctgaaccaggccccagacatgctgcatagattttatggaaagaactcttcttatgtccatgggggattggattcaaatggaaagccagcagatgcagtctacggacagaaagaaatccacaggaaagtgatgtcacaaaacttcaccaactgccacaccaagattcgccatgttgatgctcatgccacgctaaatgatggtgtggtagtccaggtgatggggcttctctctaacaacaaccaggctttgaggagattcatgcaaacgtttgtccttgctcctgaggggtctgttgcaaataaattctatgttcacaatgatatcttcagataccaagatgaggtctttggtgggtttgtcactgagcctcaggaggagtctgaagaagaagtagaggaacctgaagaaagacagcaaacacctgaggtggtacctgatgattctggaactttctatgatcaggcagttgtcagtaatgacatggaagaacatttagaggagcctgttgctgaaccagagcctgatcctgaaccagaaccagaacaagaacctgtatctgaaatccaagaggaaaagcctgagccagtattagaagaaactgcccctgaggatgctcagaagagttcttctccagcacctgcagacatagctcagacagtacaggaagacttgaggacattttcttgggcatctgtgaccagtaagaatcttccacccagtggagctgttccagttactgggataccacctcatgttgttaaagtaccagcttcacagccccgtccagagtctaagcctgaatctcagattccaccacaaagacctcagcgggatcaaagagtgcgagaacaacgaataaatattcctccccaaaggggacccagaccaatccgtgaggctggtgagcaaggtgacattgaaccccgaagaatggtgagacaccctgacagtcaccaactcttcattggcaacctgcctcatgaagtggacaaatcagagcttaaagatttctttcaaagttatggaaacgtggtggagttgcgcattaacagtggtgggaaattacccaattttggttttgttgtgtttgatgattctgagcctgttcagaaagtccttagcaacaggcccatcatgttcagaggtgaggtccgtctgaatgtcgaagagaagaagactcgagctgccagggaaggcgaccgacgagataatcgccttcggggacctggaggccctcgaggtgggctgggtggtggaatgagaggccctccccgtggaggcatggtgcagaaaccaggatttggagtgggaagggggcttgcgccacggcagtag

SEQ ID NO: 2 - DNA, 369 bp, Artificial Sequence
  Description of Artificial Sequence Synthetic polynucleotide
  G3BP1 NTF2-like domain
gtcgggcgggaatttgtgagacagtattacacactgctgaaccaggccccagacatgctgcatagattttatggaaagaactcttcttatgtccatgggggattggattcaaatggaaagccagcagatgcagtctacggacagaaagaaatccacaggaaagtgatgtcacaaaacttcaccaactgccacaccaagattcgccatgttgatgctcatgccacgctaaatgatggtgtggtagtccaggtgatggggcttctctctaacaacaaccaggctttgaggagattcatgcaaacgtttgtccttgctcctgaggggtctgttgcaaataaattctatgttcacaatgatatcttcagatac

SEQ ID NO: 3 - DNA, 228 bp, Artificial Sequence
  Description of Artificial Sequence Synthetic polynucleotide
  G3BP1 RRM
caccaactcttcattggcaacctgcctcatgaagtggacaaatcagagcttaaagatttctttcaaagttatggaaacgtggtggagttgcgcattaacagtggtgggaaattacccaattttggttttgttgtgtttgatgattctgagcctgttcagaaagtccttagcaacaggcccatcatgttcagaggtgaggtccgtctgaatgtcgaagagaagaagact

SEQ ID NO: 4 - DNA, 1032 bp, Artificial Sequence
  Description of Artificial Sequence Synthetic polynucleotide
  G3BP1 deletion NTF2-like domain
atggtgatggagaagcctagtcccctgctgcaagatgaggtctttggtgggtttgtcactgagcctcaggaggagtctgaagaagaagtagaggaacctgaagaaagacagcaaacacctgaggtggtacctgatgattctggaactttctatgatcaggcagttgtcagtaatgacatggaagaacatttagaggagcctgttgctgaaccagagcctgatcctgaaccagaaccagaacaagaacctgtatctgaaatccaagaggaaaagcctgagccagtattagaagaaactgcccctgaggatgctcagaagagttcttctccagcacctgcagacatagctcagacagtacaggaagacttgaggacattttcttgggcatctgtgaccagtaagaatcttccacccagtggagctgttccagttactgggataccacctcatgttgttaaagtaccagcttcacagccccgtccagagtctaagcctgaatctcagattccaccacaaagacctcagcgggatcaaagagtgcgagaacaacgaataaatattcctccccaaaggggacccagaccaatccgtgaggctggtgagcaaggtgacattgaaccccgaagaatggtgagacaccctgacagtcaccaactcttcattggcaacctgcctcatgaagtggacaaatcagagcttaaagatttctttcaaagttatggaaacgtggtggagttgcgcattaacagtggtgggaaattacccaattttggttttgttgtgtttgatgattctgagcctgttcagaaagtccttagcaacaggcccatcatgttcagaggtgaggtccgtctgaatgtcgaagagaagaagactcgagctgccagggaaggcgaccgacgagataatcgccttcggggacctggaggccctcgaggtgggctgggtggtggaatgagaggccctccccgtggaggcatggtgcagaaaccaggatttggagtgggaagggggcttgcgccacggcagtag

SEQ ID NO: 5 - DNA, 1173 bp, Artificial Sequence
  Description of Artificial Sequence Synthetic polynucleotide
  G3BP1 deletion RRM
atggtgatggagaagcctagtcccctgctggtcgggcgggaatttgtgagacagtattacacactgctgaaccaggccccagacatgctgcatagattttatggaaagaactcttcttatgtccatgggggattggattcaaatggaaagccagcagatgcagtctacggacagaaagaaatccacaggaaagtgatgtcacaaaacttcaccaactgccacaccaagattcgccatgttgatgctcatgccacgctaaatgatggtgtggtagtccaggtgatggggcttctctctaacaacaaccaggctttgaggagattcatgcaaacgtttgtccttgctcctgaggggtctgttgcaaataaattctatgttcacaatgatatcttcagataccaagatgaggtctttggtgggtttgtcactgagcctcaggaggagtctgaagaagaagtagaggaacctgaagaaagacagcaaacacctgaggtggtacctgatgattctggaactttctatgatcaggcagttgtcagtaatgacatggaagaacatttagaggagcctgttgctgaaccagagcctgatcctgaaccagaaccagaacaagaacctgtatctgaaatccaagaggaaaagcctgagccagtattagaagaaactgcccctgaggatgctcagaagagttcttctccagcacctgcagacatagctcagacagtacaggaagacttgaggacattttcttgggcatctgtgaccagtaagaatcttccacccagtggagctgttccagttactgggataccacctcatgttgttaaagtaccagcttcacagccccgtccagagtctaagcctgaatctcagattccaccacaaagacctcagcgggatcaaagagtgcgagaacaacgaataaatattcctccccaaaggggacccagaccaatccgtgaggctggtgagcaaggtgacattgaaccccgaagaatggtgagacaccctgacagtcgagctgccagggaaggcgaccgacgagataatcgccttcggggacctggaggccctcgaggtgggctgggtggtggaatgagaggccctccccgtggaggcatggtgcagaaaccaggatttggagtgggaagggggcttgcgccacggcagtag

SEQ ID NO: 6 - DNA, 1401 bp, Artificial Sequence
  Description of Artificial Sequence Synthetic polynucleotide
  G3BP1 pospho Ser149A
atggtgatggagaagcctagtcccctgctggtcgggcgggaatttgtgagacagtattacacactgctgaaccaggccccagacatgctgcatagattttatggaaagaactcttcttatgtccatgggggattggattcaaatggaaagccagcagatgcagtctacggacagaaagaaatccacaggaaagtgatgtcacaaaacttcaccaactgccacaccaagattcgccatgttgatgctcatgccacgctaaatgatggtgtggtagtccaggtgatggggcttctctctaacaacaaccaggctttgaggagattcatgcaaacgtttgtccttgctcctgaggggtctgttgcaaataaattctatgttcacaatgatatcttcagataccaagatgaggtctttggtgggtttgtcactgagcctcaggaggaggctgaagaagaagtagaggaacctgaagaaagacagcaaacacctgaggtggtacctgatgattctggaactttctatgatcaggcagttgtcagtaatgacatggaagaacatttagaggagcctgttgctgaaccagagcctgatcctgaaccagaaccagaacaagaacctgtatctgaaatccaagaggaaaagcctgagccagtattagaagaaactgcccctgaggatgctcagaagagttcttctccagcacctgcagacatagctcagacagtacaggaagacttgaggacattttcttgggcatctgtgaccagtaagaatcttccacccagtggagctgttccagttactgggataccacctcatgttgttaaagtaccagcttcacagccccgtccagagtctaagcctgaatctcagattccaccacaaagacctcagcgggatcaaagagtgcgagaacaacgaataaatattcctccccaaaggggacccagaccaatccgtgaggctggtgagcaaggtgacattgaaccccgaagaatggtgagacaccctgacagtcaccaactcttcattggcaacctgcctcatgaagtggacaaatcagagcttaaagatttctttcaaagttatggaaacgtggtggagttgcgcattaacagtggtgggaaattacccaattttggttttgttgtgtttgatgattctgagcctgttcagaaagtccttagcaacaggcccatcatgttcagaggtgaggtccgtctgaatgtcgaagagaagaagactcgagctgccagggaaggcgaccgacgagataatcgccttcggggacctggaggccctcgaggtgggctgggtggtggaatgagaggccctccccgtggaggcatggtgcagaaaccaggatttggagtgggaagggggcttgcgccacggcagtag

SEQ ID NO: 7 - DNA, 1401 bp, Artificial Sequence
  Description of Artificial Sequence Synthetic polynucleotide
  G3BP1 pospho Ser149D
atggtgatggagaagcctagtcccctgctggtcgggcgggaatttgtgagacagtattacacactgctgaaccaggccccagacatgctgcatagattttatggaaagaactcttcttatgtccatgggggattggattcaaatggaaagccagcagatgcagtctacggacagaaagaaatccacaggaaagtgatgtcacaaaacttcaccaactgccacaccaagattcgccatgttgatgctcatgccacgctaaatgatggtgtggtagtccaggtgatggggcttctctctaacaacaaccaggctttgaggagattcatgcaaacgtttgtccttgctcctgaggggtctgttgcaaataaattctatgttcacaatgatatcttcagataccaagatgaggtctttggtgggtttgtcactgagcctcaggaggaggatgaagaagaagtagaggaacctgaagaaagacagcaaacacctgaggtggtacctgatgattctggaactttctatgatcaggcagttgtcagtaatgacatggaagaacatttagaggagcctgttgctgaaccagagcctgatcctgaaccagaaccagaacaagaacctgtatctgaaatccaagaggaaaagcctgagccagtattagaagaaactgcccctgaggatgctcagaagagttcttctccagcacctgcagacatagctcagacagtacaggaagacttgaggacattttcttgggcatctgtgaccagtaagaatcttccacccagtggagctgttccagttactgggataccacctcatgttgttaaagtaccagcttcacagccccgtccagagtctaagcctgaatctcagattccaccacaaagacctcagcgggatcaaagagtgcgagaacaacgaataaatattcctccccaaaggggacccagaccaatccgtgaggctggtgagcaaggtgacattgaaccccgaagaatggtgagacaccctgacagtcaccaactcttcattggcaacctgcctcatgaagtggacaaatcagagcttaaagatttctttcaaagttatggaaacgtggtggagttgcgcattaacagtggtgggaaattacccaattttggttttgttgtgtttgatgattctgagcctgttcagaaagtccttagcaacaggcccatcatgttcagaggtgaggtccgtctgaatgtcgaagagaagaagactcgagctgccagggaaggcgaccgacgagataatcgccttcggggacctggaggccctcgaggtgggctgggtggtggaatgagaggccctccccgtggaggcatggtgcagaaaccaggatttggagtgggaagggggcttgcgccacggcagtag

SEQ ID NO: 8 - DNA, 35 bp, Artificial Sequence
  Description of Artificial Sequence Synthetic oligonucleotide
  Primers
ctgagcctcaggaggaggctgaagaagaagtagag

SEQ ID NO: 9 - DNA, 35 bp, Artificial Sequence
  Description of Artificial Sequence Synthetic oligonucleotide
  Primers
ctctacttcttcttcagcctcctcctgaggctcag

SEQ ID NO: 10 - DNA, 35 bp, Artificial Sequence
  Description of Artificial Sequence Synthetic oligonucleotide
  Primers
ctgagcctcaggaggaggatgaagaagaagtagag

SEQ ID NO: 11 - DNA, 35 bp, Artificial Sequence
  Description of Artificial Sequence Synthetic oligonucleotide
  Primers
ctctacttcttcttcatcctcctcctgaggctcag

SEQ ID NO: 12 - DNA, 20 bp, Artificial Sequence
  Description of Artificial Sequence Synthetic oligonucleotide
  Primers
gaaatccaagaggaaaagcc

SEQ ID NO: 13 - DNA, 20 bp, Artificial Sequence
  Description of Artificial Sequence Synthetic oligonucleotide
  Primers
cccaagaaaatgtcctcaag

SEQ ID NO: 14 - DNA, 18 bp, Artificial Sequence
  Description of Artificial Sequence Synthetic oligonucleotide
  Primers
acagttgccatgtagacc

SEQ ID NO: 15 - DNA, 19 bp, Artificial Sequence
  Description of Artificial Sequence Synthetic oligonucleotide
  Primers
ttgagcacagggtacttta

SEQ ID NO: 16 - DNA, 20 bp, Artificial Sequence
  Description of Artificial Sequence Synthetic oligonucleotide
  Primers
agggatttgaatcacgtttg

SEQ ID NO: 17 - DNA, 20 bp, Artificial Sequence
  Description of Artificial Sequence Synthetic oligonucleotide
  Primers
tttactggcaacatcaacag